(19) United States
(12) Patent Application Publication
(10) Pub. No.: US 2022/0193238 A1
(43) Pub. Date: Jun. 23, 2022

(54) ANTI-IL5R ANTIBODY FORMULATIONS

(71) Applicant: ASTRAZENECA AB, Sodertalje (SE)

(72) Inventors: SABA GHAZVINI, GAITHERSBURG, MD (US); CASSANDRA MANKUS, GAITHERSBURG, MD (US)

(21) Appl. No.: 17/552,383

(22) Filed: Dec. 16, 2021

Related U.S. Application Data
(60) Provisional application No. 63/199,277, filed on Dec. 17, 2020.

(51) Int. Cl. A61K 39/395; C07K 16/28; A61K 47/26; A61K 47/34; A61K 47/22; A61K 47/14

(52) U.S. Cl. CPC A61K 39/39591; C07K 16/2866; A61K 47/14; A61K 47/34; A61K 47/22; A61K 47/26

(57) ABSTRACT

Provided herein are anti-IL5R antibody formulations containing an amount of surfactant below critical micelle concentration (CMC) of the surfactant and methods of using such formulations.

### CROSS REFERENCE TO RELATED APPLICATIONS

This application claims the priority benefit of U.S. Provisional Application No. 63/199,277, filed Dec. 17, 2020, which is hereby incorporated by reference herein in its entirety.

### REFERENCE TO SEQUENCE LISTING SUBMITTED ELECTRONICALLY VIA EFS-WEB

The content of the electronically submitted sequence listing (Name: IL5R-301-WO-PCT_ST25.txt, Size: 9,485 bytes; and Date of Creation: Nov. 19, 2021) submitted in this application is incorporated herein by reference in its entirety.

### FIELD OF DISCLOSURE

The present disclosure relates to anti-IL5R antibody formulations containing an amount of surfactant below critical micelle concentration (CMC) of the surfactant and methods of using such formulations.

### BACKGROUND

Antibodies have been used in the treatment of various diseases and conditions due to their specificity of target recognition, thereby generating highly selective outcomes following systemic administration. In order for antibodies to remain effective, they must maintain their biological activity during their production, purification, transport and storage. New production and purification techniques have been developed to provide for large amounts of highly purified monoclonal antibodies to be produced. However, challenges still exist to stabilize these antibodies for transport and storage, and yet even more challenges exist to provide the antibodies in a dosage form suitable for administration.

Denaturation, aggregation, contamination, and particle formation can be significant obstacles in the formulation and storage of antibodies. Due to the wide variety of antibodies, there are no universal formulations or conditions suitable for storage of all antibodies. Optimal formulations and conditions suitable for storage of one antibody are often specific to that antibody. Thus, antibody storage formulations and methods are often a significant part of the research and development process for a commercial antibody.

Various methods have been proposed to overcome the challenges associated with antibody stability. For example, in some instances, the antibody can be lyophilized, and then reconstituted shortly before administration. However, reconstitution may not be ideal, since it adds an additional step to the administration process, and could introduce contaminants to the formulation. Additionally, even reconstituted antibodies can suffer from aggregation and particle formation. Thus, a need exists to provide stable antibody formulations that can overcome the challenges associated with transport and storage.

### SUMMARY OF DISCLOSURE

Provided herein are anti-IL5R antibody formulations containing an amount of surfactant below critical micelle concentration (CMC) of the surfactant. In some aspects, the formulation comprises (i) an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; and a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and (ii) a surfactant at an amount below CMC of the surfactant, wherein the surfactant is not polysorbate 20 (PS20). In some aspects, the surfactant is polysorbate 80 (PS80), poloxamer, a Brij series surfactant, or tocopheryl polyethylene glycol succinate (TPGS).

In some aspects, the formulation comprises (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and (ii) about 0.0004% (w/v) to about 0.0012% (w/v) PS80, or about 0.03% (w/v) to about 0.08% (w/v) poloxamer, or about 0.001% (w/v) to about 0.02% (w/v) tocopheryl polyethylene glycol succinate (TPGS), or about 0.001% (w/v) to about 0.01% (w/v) of a Brij series surfactant.

In some aspects, the formulation comprises (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0004% (w/v) PS80; (iii) about 250 mM trehalose; (iv) about 130 mM L-arginine; and (v) about 20 mM histidine.

In some aspects, the formulation comprises (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0012% (w/v) PS80; (iii) about 250 mM trehalose; (iv) about 130 mM L-arginine; and (v) about 20 mM histidine.

In some aspects, the formulation comprises (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.03% (w/v) poloxamer; (iii) about 250 mM trehalose; (iv) about 130 mM L-arginine; and (v) about 20 mM histidine.

In some aspects, the formulation comprises (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.08% (w/v) poloxamer; (iii) about 250 mM trehalose; (iv) about 130 mM L-arginine; and (v) about 20 mM histidine.

In some aspects, the formulation comprises (i) about 30 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0004% (w/v) PS80; (iii) about 250 mM trehalose; (iv) about 130 mM L-arginine; and (v) about 20 mM histidine.

In some aspects, the formulation comprises (i) about 30 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0012% (w/v) PS80; (iii) about 250 mM trehalose; (iv) about 130 mM L-arginine; and (v) about 20 mM histidine.

In some aspects, the formulation comprises (i) about 30 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.03% (w/v) poloxamer 188; (iii) about 250 mM trehalose; (iv) about 130 mM L-arginine; and (v) about 20 mM histidine.

In some aspects, the formulation comprises (i) about 30 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO: 5; and a light chain variable region CDR3 comprising the sequence set forth in SEQ ID NO:6; (ii) about 0.08% (w/v) poloxamer 188; (iii) about 250 mM trehalose; (iv) about 130 mM L-arginine; and (v) about 20 mM histidine.

In some aspects, a pharmaceutical formulation, comprises: (i) about 2 mg/mL to about 200 mg/mL (e.g., about 150 mg/mL or about 30 mg/mL) of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer; (iii) about 250 mM sucrose; and (iv) acetate.

In some aspects, a pharmaceutical formulation, comprises: (i) about 2 mg/mL to about 200 mg/mL (e.g., about 150 mg/mL or about 30 mg/mL) of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer; (iii) about 250 mM sucrose; and (iv) succinate.

In some aspects, a pharmaceutical formulation, comprises: (i) about 2 mg/mL to about 200 mg/mL (e.g., about 150 mg/mL or about 30 mg/mL) of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer; (iii) about 250 mM mannitol; and (iv) acetate.

In some aspects, a pharmaceutical formulation, comprises: (i) about 2 mg/mL to about 200 mg/mL (e.g., about 150 mg/mL or about 30 mg/mL) of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer; (iii) about 250 mM mannitol; and (iv) succinate.

Also provided herein is a dosage form comprising an anti-IL5R antibody formulation provided herein in a container. In some aspects, the container is a vial or a syringe. In some aspects, the vial is glass or plastic. In some aspects, the syringe is a prefilled syringe. In some aspects, the syringe is plastic or glass. In some aspects, the syringe comprises a needle.

Also provided herein is a kit comprising an anti-IL5R antibody formulation provided herein, a dosage form provided herein, a vial provided herein, or a syringe provided herein, and instructions for use.

Also provided herein is a method of treating a pulmonary disease or disorder in a subject, comprising administering to the subject a therapeutically effective amount of an anti-IL5R antibody formulation provided herein, a dosage form provided herein, a vial provided herein, or a syringe provided herein. In some aspects, the pulmonary disease or disorder is an eosinophilic disease or disorder. In some aspects, the pulmonary disease or disorder is asthma, chronic obstructive pulmonary disease (COPD), allergic bronchopulmonary aspergillosis, acute and chronic eosinophilic bronchitis, acute and chronic eosinophilic pneumonia, Churg-Strauss syndrome, hypereosinophilic syndrome, drug, irritant and radiation-induced pulmonary eosinophilia, infection-induced pulmonary eosinophilia, autoimmune-related pulmonary eosinophilia, eosinophilic esophagitis, Crohn's disease, or a combination thereof. In some aspects, the asthma is eosinophilic asthma, neutrophilic asthma, combined eosinophilic and neutrophilic asthma, or aspirin sensitive asthma.

### BRIEF DESCRIPTION OF FIGURES

FIGS. 1A-1B. FIG. 1A shows the average number of subvisible particles (≥2 μm) per mL of different anti-IL5R antibody formulations, contained in a prefilled syringe and subjected to aggressive agitation (end-to-end rotation) for 5 or 10 days or no agitation (control). The formulations contained either no surfactant, an amount of surfactant above critical micelle concentration (CMC) [i.e., 0.02% polysorbate 20 (PS20), 0.006% polysorbate 80 (PS80), and 0.02% PS80], or an amount of surfactant below CMC [i.e., 0.006% PS20, 0.0004% PS80, 0.0012% PS80, 0.027% poloxamer 188 (poloxamer), and 0.08% poloxamer]. FIG. 1B shows the average number of subvisible particles (≥25 μm) per mL of different anti-IL5R antibody formulations, contained in a prefilled syringe and subjected to aggressive agitation (end-to-end rotation) for 5 or 10 days or no agitation (control). The formulations contained either no surfactant, an amount of surfactant above CMC (i.e., 0.02% PS20, 0.006% PS80, and 0.02% PS80), or an amount of surfactant below CMC (i.e., 0.006% PS20, 0.0004% PS80, 0.0012% PS80, 0.027% poloxamer, and 0.08% poloxamer).

FIGS. 2A-2D. FIG. 2A shows the average number of subvisible particles (≥22 μm) per mL of different anti-IL5R antibody formulations, contained in a glass vial and subjected to aggressive agitation (end-to-end rotation) for 5 or 10 days or no agitation (control). The formulations contained either no surfactant, an amount of surfactant above critical micelle concentration (CMC) [i.e., 0.02% polysorbate 20 (PS20), 0.006% polysorbate 80 (PS80), and 0.02% PS80], or an amount of surfactant below CMC [i.e., 0.006% PS20, 0.0004% PS80, 0.0012% PS80, 0.027% poloxamer 188 (poloxamer), and 0.08% poloxamer]. FIG. 2B shows the average number of subvisible particles (≥5 μm) per mL of different anti-IL5R antibody formulations, contained in a glass vial and subjected to aggressive agitation (end-to-end rotation) for 5 or 10 days or no agitation (control). The formulations contained either no surfactant, an amount of surfactant above CMC (i.e., 0.02% PS20, 0.006% PS80, and 0.02% PS80), or an amount of surfactant below CMC (i.e., 0.006% PS20, 0.0004% PS80, 0.0012% PS80, 0.027% poloxamer, and 0.08% poloxamer). FIG. 2C shows the average number of subvisible particles (≥10 μm) per mL of different anti-IL5R antibody formulations, contained in a glass vial and subjected to aggressive agitation (end-to-end rotation) for 5 or 10 days or no agitation (control). The formulations contained either no surfactant, an amount of surfactant above CMC (i.e., 0.02% PS20, 0.006% PS80, and 0.02% PS80), or an amount of surfactant below CMC (i.e., 0.006% PS20, 0.0004% PS80, 0.0012% PS80, 0.027% poloxamer, and 0.08% poloxamer). FIG. 2D shows the average number of subvisible particles (≥25 μm) per mL of different anti-IL5R antibody formulations, contained in a glass vial and subjected to aggressive agitation (end-to-end rotation) for 5 or 10 days or no agitation (control). The formulations contained either no surfactant, an amount of surfactant above CMC (i.e., 0.02% PS20, 0.006% PS80, and 0.02% PS80), or an amount of surfactant below CMC (i.e., 0.006% PS20, 0.0004% PS80, 0.0012% PS80, 0.027% poloxamer, and 0.08% poloxamer).

FIGS. 3A-3B. FIG. 3A shows the average number of subvisible particles (≥22 μm) per mL of different anti-IL5R antibody formulations, contained in a prefilled syringe and subjected to seven freeze-thaw cycles (7×FT) or no freeze-thaw cycles (control). The formulations contained either no surfactant, an amount of surfactant above CMC (i.e., 0.02% PS20, 0.006% PS80, and 0.02% PS80), or an amount of surfactant below CMC (i.e., 0.006% PS20, 0.0004% PS80, 0.0012% PS80, 0.027% poloxamer, and 0.08% poloxamer). FIG. 3B shows the average number of subvisible particles (≥25 μm) per mL of different anti-IL5R antibody formulations, contained in a prefilled syringe and subjected to seven freeze-thaw cycles (7×FT) or no freeze-thaw cycles (control). The formulations contained either no surfactant, an amount of surfactant above CMC (i.e., 0.02% PS20, 0.006% PS80, and 0.02% PS80), or an amount of surfactant below CMC (i.e., 0.006% PS20, 0.0004% PS80, 0.0012% PS80, 0.027% poloxamer, and 0.08% poloxamer).

FIGS. 4A-4B. FIG. 4A shows the average number of subvisible particles (≥1 μm, ≥22 μm and ≥5 μm) per mL of different anti-IL5R antibody formulations. The formulations contained either no surfactant or an amount of surfactant below CMC (i.e., 0.0004% PS80, 0.0012% PS80, 0.027% poloxamer, and 0.08% poloxamer). FIG. 4B shows the average number of subvisible particles (≥10 μm and ≥25 μm) per mL of different anti-IL5R antibody formulations. The formulations contained either no surfactant or an amount of surfactant below CMC (i.e., 0.0004% PS80, 0.0012% PS80, 0.027% poloxamer, and 0.08% poloxamer).

FIGS. 5A-5B. FIG. 5A shows the average number of subvisible particles (≥1 μm, ≥22 μm and ≥5 μm) per mL of different anti-IL5R antibody formulations. The formulations contained either no surfactant, an amount of PS80 above CMC (i.e., 0.006% and 0.02%), or an amount of PS80 below CMC (i.e., 0.0004% and 0.0012%). FIG. 5B shows the average number of subvisible particles (≥10 μm and ≥25 μm) per mL of different anti-IL5R antibody formulations. The formulations contained either no surfactant, an amount of PS80 above CMC (i.e., 0.006% and 0.02%), or an amount of PS80 below CMC (i.e., 0.0004% and 0.0012%).

FIG. 6 shows the average number of subvisible particles (≥22 μm) per mL of different anti-IL5R antibody formulations contained in a vial or prefilled syringe. The formulations contained either no surfactant, an amount of surfactant above CMC (e.g., 0.004% PS80, 0.01% PS80, 0.27% poloxamer, and 0.67% poloxamer), or an amount of surfactant below CMC (e.g., 0.0004% PS80, 0.0012% PS80, 0.027% poloxamer, and 0.08% poloxamer).

FIG. 7 shows the average number of subvisible particles (≥22 μm) per mL of different anti-IL5R antibody formulations contained in a vial, a prefilled syringe containing silicone, or a prefilled syringe not containing silicone. The formulations contained either an amount of surfactant above CMC (i.e., 0.02% PS20, 0.05% PS20, 0.004% PS80, and 0.01% PS80) or an amount of surfactant below CMC (i.e., 0.0004% PS80, 0.0012% PS80, 0.002% PS20, and 0.006% PS20).

### DETAILED DESCRIPTION OF DISCLOSURE

It should be appreciated that the particular implementations shown and described herein are examples, and are not intended to otherwise limit the scope of the application in any way. It should also be appreciated that each of the aspects and features described herein can be combined in any and all ways.

Furthermore, the published patents, patent applications, websites, company names, and scientific literature referred to herein are hereby incorporated by reference in their entirety to the same extent as if each was specifically and individually indicated to be incorporated by reference.

In a first aspect (A1) provided herein is a pharmaceutical formulation, comprising: (i) an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; and a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and (ii) a surfactant at an amount below the critical micelle concentration (CMC) of said surfactant, wherein the surfactant is not polysorbate 20 (PS20). In a another aspect (A2) of A1, the surfactant is polysorbate 80 (PS80), poloxamer, a Brij series surfactant, or tocopheryl polyethylene glycol succinate (TPGS). In another aspect (A3) of A1 or A2, the antibody or antigen binding fragment thereof comprises a heavy chain variable region comprising SEQ ID NO:7 and a light chain variable region comprising SEQ ID NO:8. In another aspect (A4) of any one of A1-A3, the antibody or antigen binding fragment thereof comprises a heavy chain comprising SEQ ID NO:9 and a light chain comprising SEQ ID NO:10. In another aspect (A5) of any one of A1-A4, the pharmaceutical formulation comprises from about 2 mg/mL to about 100 mg/mL of the antibody or antigen binding fragment thereof. In another aspect (A6) of any one of A1-A5, the pharmaceutical formulation comprises about 30 mg/mL of the antibody or antigen binding fragment thereof. In another aspect (A7) of any one of A1-A6, the amount of surfactant is at least about 10% below, at least 20% below, at least 30% below, at least 40% below, at least 50% below, at least 60% below, at least 70% below, at least 80% below, at least 90% below, at least 95% below, at least 96% below, at least 97% below, at least 98% below, or at least 99% below the CMC of said surfactant. In another aspect (A8) of any one of A1-A7, the surfactant is PS80 at an amount of from about 0.0004% (w/v) to about 0.0012% (w/v). In another aspect (A9) of any one of A1-A7, the surfactant is TPGS at an amount of from about 0.001% (w/v) to about 0.02% (w/v). In another aspect (A10) of any one of A1-A7, the surfactant is a Brij series surfactant at an amount of from about 0.001% (w/v) to about 0.01% (w/v). In another aspect (A11) of any one of A1-A7, the surfactant is poloxamer at an amount of from about 0.03% (w/v) to about 0.08% (w/v). In another aspect (A12), the poloxamer is poloxamer 188.

In another aspect (A13) of any one of A1-A11, the pharmaceutical formulation further comprises (iii) an uncharged excipient. In another aspect (A14) of A13, the pharmaceutical formulation comprises from about 20 mM to about 80 mM of the uncharged excipient. In another aspect (A15) of A13, the pharmaceutical formulation comprises from about 200 mM to about 400 mM of the uncharged excipient. In another aspect (A16) of any one of A13-A15, the pharmaceutical formulation comprises from about 1.5% (w/v) to about 8.5% (w/v) of the uncharged excipient. In another aspect (A17) of any one of A13-A16, the uncharged excipient is fructose, glucose, mannose, sorbose, xylose, lactose, maltose, sucrose, dextran, pullulan, dextrin, cyclodextrin, soluble starch, trehalose, sorbitol, erythritol, isomalt, lactitol, maltitol, xylitol, glycerol, lactitol, hydroxyethyl starch, water-soluble glucan, or a combination thereof. In another aspect (A18) of A17, the uncharged excipient is trehalose.

In another aspect (A19) of any one of A1-A18, the pharmaceutical formulation further comprises (iv) arginine. In another aspect (A20) of A19, the pharmaceutical formulation comprises from about 100 mM to about 200 mM arginine. In another aspect (A21) of A19 or A20, the arginine is L-arginine. In another aspect (A22) of any one of A19-A21, the pharmaceutical formulation comprises from about 120 mM to about 140 mM L-arginine and from about 40 mM to about 60 mM uncharged excipient.

In another aspect (A23) of any one of A1-A22, the pharmaceutical composition further comprises (v) histidine. In another aspect (A24) of A23, the pharmaceutical composition comprises from about 15 mM to about 30 mM histidine.

In another aspect (A25) provided herein is a pharmaceutical formulation, comprising: (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO: 5; and a light chain variable region CDR3 comprising the sequence set forth in SEQ ID NO:6; and (ii) about 0.0004% (w/v) to about 0.0012% (w/v) PS80, or about 0.03% (w/v) to about 0.08% (w/v) poloxamer.

In another aspect (A26) of A25, the pharmaceutical formulation further comprises (iii) about 1.5% (w/v) to about 8.5% (w/v) trehalose; (iv) about 100 mM to about 200 mM L-arginine; and (v) about 15 mM to about 30 mM histidine.

In another aspect (A27) provided herein is a pharmaceutical formulation, comprising: (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ TD NO:5; and a light chain variable region CDR3 comprising the sequence set forth in SEQ ID NO:6; (ii) about 0.0004% (w/v) PS80; (iii) about 250 mM trehalose; (iv) about 130 mM L-arginine; and (v) about 20 mM histidine.

In another aspect (A28) provided herein is a pharmaceutical formulation, comprising: (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO: 5; and a light chain variable region CDR3 comprising the sequence set forth in SEQ ID NO:6; (ii) about 0.0012% (w/v) PS80; (iii) about 250 mM trehalose; (iv) about 130 mM L-arginine; and (v) about 20 mM histidine.

In another aspect (A29) provided herein is a pharmaceutical formulation, comprising: (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.03% (w/v) poloxamer; (iii) about 250 mM trehalose; (iv) about 130 mM L-arginine; and (v) about 20 mM histidine.

In another aspect (A30) provided herein is a pharmaceutical formulation, comprising: (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ TD NO:5; and a light chain variable region CDR3 comprising the sequence set forth in SEQ ID NO:6; (ii) about 0.08% (w/v) poloxamer; (iii) about 250 mM trehalose; (iv) about 130 mM L-arginine; and (v) about 20 mM histidine.

In another aspect (A31) provided herein is a pharmaceutical formulation, comprising: (i) about 30 mg/mL of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0004% (w/v) PS80; (iii) about 250 mM trehalose; (iv) about 130 mM L-arginine; and (v) about 20 mM histidine.

In another aspect (A32) provided herein is a pharmaceutical formulation, comprising: (i) about 30 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0012% (w/v) PS80; (iii) about 250 mM trehalose; (iv) about 130 mM L-arginine; and (v) about 20 mM histidine.

In another aspect (A33) provided herein is a pharmaceutical formulation, comprising: (i) about 30 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.03% (w/v) poloxamer 188; (iii) about 250 mM trehalose; (iv) about 130 mM L-arginine; and (v) about 20 mM histidine.

In another aspect (A34) provided herein is a pharmaceutical formulation, comprising: (i) about 30 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.08% (w/v) poloxamer 188; (iii) about 250 mM trehalose; (iv) about 130 mM L-arginine; and (v) about 20 mM histidine.

In another aspect (A35) of any one of A1-A34, the pharmaceutical formulation is stable following aggressive agitation for about 10 days at room temperature. In another aspect (A36) of A35, the pharmaceutical formulation has less than 20 subvisible particles per mL.

In another aspect (A37) of any one of A1-A36, the pharmaceutical formulation has about 20% less, about 30% less, about 40% less, about 50% less, about 60% less, about 70% less, about 80% less, about 90% less, about 95% less, or about 99% less subvisible particles following from about 1 to about 7 freeze-thaw cycles.

In another aspect (A38) of any one of A1-A37, the pharmaceutical formulation has about 20% less, about 30% less, about 40% less, about 50% less, about 60% less, about 70% less, about 80% less, about 90% less, about 95% less, or about 99% less subvisible particles following storage for about 1 month at about 40° C.

In another aspect (A39) of any one of A1-A38, the pharmaceutical formulation is suitable for intravenous, subcutaneous, or intramuscular administration. In another aspect (A40) of any one of A1-A39, the pharmaceutical formulation is a liquid pharmaceutical formulation. In another aspect (A41) of any one of A1-A39, the pharmaceutical formulation is a lyophilized pharmaceutical formulation.

In another aspect (A42) provided herein is a dosage form comprising the pharmaceutical formulation of any one of A1-A41 in a container. In another aspect (A43) of A42, the container is a plastic vial or glass vial. In another aspect (A44) of A42, the container is a pre-filled syringe. In another aspect (A45) of A44, the pre-filled syringe comprises a needle. In another aspect (A46) of A45, the needle is a 29 G thin wall needle. In another aspect (A47) of any one of A44-A46, the pre-filled syringe is a plastic syringe or a glass syringe. In another aspect (A48) of any one of A44-A47, the pre-filled syringe is coated with silicone.

In another aspect (A49) provided herein is a vial comprising: (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and (ii) about 0.0004% (w/v) to about 0.0012% (w/v) PS80, or about 0.03% (w/v) to about 0.08% (w/v) poloxamer. In another aspect (A50) in A49 further comprises: (iii) about 1.5% (w/v) to about 8.5% (w/v) trehalose; (iv) about 100 mM to about 200 mM L-arginine; and (v) about 15 mM to about 30 mM histidine.

In another aspect (A51), provided herein is a pre-filled syringe comprising: (i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; and a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and (ii) about 0.0004% (w/v) to about 0.0012% (w/v) PS80, or about 0.03% (w/v) to about 0.08% (w/v) poloxamer. In another aspect (A52) of A51, the pre-filled syringe further comprises: (iii) about 1.5% (w/v) to about 8.5% (w/v) trehalose; (iv) about 100 mM to about 200 mM L-arginine; and (v) about 15 mM to about 30 mM histidine.

In another aspect (A53), provided herein is a kit comprising the pharmaceutical formulation of any one of A1-A41, the dosage form of any one of A42-A48, the vial of A49 or A50, or the pre-filled syringe of A51 or A52, and instructions for use.

In another aspect (A54), provided herein is a method of treating a pulmonary disease or disorder in a subject, comprising administering to the subject a therapeutically effective amount of the pharmaceutical formulation of any one of A1-A41, the dosage form of any one of A42-A48, the vial of A49 or A50, or the pre-filled syringe of A51 or A52. In another aspect (A55) of A54, the pulmonary disease or disorder is an eosinophilic disease or disorder. In another aspect (A56) of A54, the pulmonary disease or disorder is asthma, chronic obstructive pulmonary disease (COPD), allergic bronchopulmonary aspergillosis, acute and chronic eosinophilic bronchitis, acute and chronic eosinophilic pneumonia, Churg-Strauss syndrome, hypereosinophilic syndrome, drug, irritant and radiation-induced pulmonary eosinophilia, infection-induced pulmonary eosinophilia, autoimmune-related pulmonary eosinophilia, eosinophilic esophagitis, Crohn's disease, or a combination thereof. In another aspect (A57) of A56, the pulmonary disease or disorder is asthma. In another aspect (A58) of A57, the asthma is eosinophilic asthma, neutrophilic asthma, combined eosinophilic and neutrophilic asthma, or aspirin sensitive asthma.

### I. Definitions

In order that the present description can be more readily understood, certain terms are first defined. Additional definitions are set forth throughout the detailed description.

It is to be noted that the term “a” or “an” entity refers to one or more of that entity; for example, “an anti-IL5R antibody” is understood to represent one or more anti-IL5R antibodies. As such, the terms “a” (or “an”), “one or more,” and “at least one” can be used interchangeably herein.

Throughout the present disclosure, all expressions of percentage, ratio, and the like are “by weight” unless otherwise indicated. As used herein, “by weight” is synonymous with the term “by mass,” and indicates that a ratio or percentage defined herein is done according to weight rather than volume, thickness, or some other measure.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Technical and scientific terms used herein have the meaning commonly understood by one of skilled in the art to which the present disclosure pertains, unless otherwise defined. Reference is made herein to various methodologies and materials known to those of skilled in the art. Standard reference works setting forth the general principles of recombinant DNA technology include Sambrook et al., “Molecular Cloning: A Laboratory Manual,” 2nd Ed., Cold Spring Harbor Laboratory Press, New York (1989); Kaufman et al., Eds., “Handbook of Molecular and Cellular Methods in Biology in Medicine,” CRC Press, Boca Raton (1995); and McPherson, Ed., “Directed Mutagenesis: A Practical Approach,” IRL Press, Oxford (1991), the disclosures of each of which are incorporated by reference herein in their entireties.

### II. Formulations and Related Aspects

The present disclosure is directed to anti-IL5 receptor (anti-IL5R) antibody formulations containing an amount of surfactant below critical micelle concentration (CMC) of the surfactant. In some aspects, the surfactant is not polysorbate 20 (PS20).

As described herein, the term “anti-IL5R antibody formulation” refer to a composition comprising one or more anti-IL5R antibody molecules or one or more antigen binding fragments thereof. The term “antibody” is not particularly limited. An “antibody” is taken in its broadest sense and includes any immunoglobulin (Ig), active or desired variants thereof. The term “antibody” can also refer to dimers or multimers. An antibody can be polyclonal or monoclonal and can be naturally-occurring or recombinantly-produced. Thus, human, non-human, humanized, and chimeric antibodies are all included with the term “antibody.” Typically an antibody is a monoclonal antibody of one of the following classes: IgG, IgE, IgM, IgD, and IgA; and more typically is an IgG or IgA.

An antibody can be from any animal origin including birds and mammals. In some aspects, an antibody is from human, murine (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins. See, e.g., U.S. Pat. No. 5,939,598, which is incorporated by reference herein in its entirety.

An antibody can also include, e.g., native antibodies, intact monoclonal antibodies, polyclonal antibodies, multispecific antibodies (e.g., bispecific antibodies) formed from at least two intact antibodies, antibody fragments (e.g., antibody fragments that bind to and/or recognize one or more antigens), humanized antibodies, human antibodies [Jakobovits et al., Proc. Natl. Acad. Sci. USA 90:2551 (1993); Jakobovits et al., Nature 362:255-258 (1993); Bruggermann et al., Year in Immunol. 7:33 (1993); U.S. Pat. Nos. 5,591,669 and 5,545,807), antibodies and antibody fragments isolated from antibody phage libraries (McCafferty et al., Nature 348:552-554 (1990); Clackson et al., Nature 352:624-628 (1991); Marks et al., J. Mol. Biol. 222:581-597 (1991); Marks et al., Bio/Technology 10:779-783 (1992); Waterhouse et al., Nucl. Acids Res. 21:2265-2266 (1993)].

An anti-IL5R antibody immunospecifically binds to an interleukin-5 (IL5) receptor (IL5R) polypeptide and does not specifically bind to other polypeptides. Preferably, antibodies or antigen binding fragments thereof that immunospecifically bind to an IL5R have a higher affinity to an IL5R or a fragment thereof when compared to the affinity to other polypeptides or fragments of other polypeptides. That is, antibodies or antigen binding fragments thereof that immunospecifically bind to an IL5R or fragment thereof with a higher energy than to other polypeptides or fragments of other polypeptides (see, e.g., Paul ed., 1989, Fundamental Immunology, 2^{nd} ed., Raven Press, New York at pages 332-336 for a discussion regarding antibody specificity).

Antibodies or antigen binding fragments thereof that immunospecifically bind to an IL5R can be identified, for example, by immunoassays such as radioimmunoassays (RIAs), enzyme-linked immunosorbent assays (ELISAs), and BIAcore assays or other techniques known to those of skill in the art (see, e.g., Seymour et al, 1995, Immunology—An Introduction for the Health Sciences, McGraw-Hill Book Company, Australia at pages 33-41 for a discussion of various assays to determine antibody-antigen interactions in vivo).

In one aspect, an IL5R is human IL5R, an analog, derivative or a fragment thereof. The nucleotide sequence of human IL5R is found in the GenBank database (see, e.g., Accession No. M96652.1). The amino acid sequence of human IL5R is found in the GenBank database (see, e.g., Accession No. Q01344). These sequences are incorporated by reference herein.

In some aspects, an anti-IL5R antibody or antigen binding fragment thereof is benralizumab. Information regarding benralizumab and antigen binding fragments thereof is found, for example, in U.S. Patent Application Publication No. 2010/0291073, which is incorporated herein by reference in its entirety. In some aspects, benralizumab or antigen binding fragments thereof comprise the variable heavy chain and variable light chain CDR sequences of the KM1259 antibody as disclosed in U.S. Pat. No. 6,018,032, which is incorporated herein by reference in its entirety.

In some aspects, an anti-IL5R antibody or antigen binding fragment thereof comprises a heavy chain variable region CDR1 comprising the sequence set forth in SEQ ID NO:1; a heavy chain variable region CDR2 comprising the sequence set forth in SEQ ID NO:2; and a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO: 5; and/or a light chain variable region CDR3 comprising the sequence set forth in SEQ ID NO:6.

In some aspects, an anti-IL5R antibody or antigen binding fragment thereof comprises a heavy chain variable region comprising SEQ ID NO:7 and/or a light chain variable region comprising SEQ ID NO:8.

In some aspects, an anti-IL5R antibody or antigen binding fragment thereof comprises a heavy chain comprising SEQ ID NO:9 and/or a light chain comprising SEQ ID NO:10.

In some aspects, an anti-IL5R antibody or antigen binding fragment is present in the formulation at an amount of from about 1 mg/ml to about 200 mg/ml, about 1 mg/ml to about 100 mg, about 1 mg/ml to about 50 mg, about 2 mg/ml to about 100 mg/ml, about 2 mg/ml to about 30 mg/ml, about 2 mg/ml to about 25 mg/ml, about 2 mg/ml to about 20 mg/ml, about 3 mg/ml, about 4 mg/ml, about 5 mg/ml, about 6 mg/ml, about 7 mg/ml, about 8 mg/ml, about 9 mg/m, about 10 mg/ml, about 11 mg/ml, about 12 mg/m, about 13 mg/ml, about 14 mg/ml, about 15 mg/ml, about 16 mg/ml, about 17 mg/ml, about 18 mg/ml, about 19 mg/ml, about 20 mg/ml, about 25 mg/ml, about 30 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, or about 100 mg/ml. In some aspects, an anti-IL5R antibody or antigen binding fragment is present in the formulation at an amount of from about 2 mg/ml to about 200 mg/ml, about 30 mg/ml to about 200 mg/ml. In some aspects, an anti-IL5R antibody or antigen binding fragment is present in the formulation at an amount of from about 2 mg/ml to about 150 mg/ml, about 30 mg/ml to about 150 mg/ml. In some aspects, an anti-IL5R antibody or antigen binding fragment is present in the formulation at about 150 mg/ml.

Anti-IL5R antibody formulations of the present disclosure contain an amount of surfactant below critical micelle concentration (CMC) of the surfactant. As used herein, “critical micelle concentration” or “CMC” is a concentration of surfactant at or above which micelles form and all additional surfactants added to the system will form micelles. The CMC value(s) of a surfactant can be calculated from different known techniques (e.g., surface tensiometry, conductivity, light scattering, fluorescence spectroscopy, and/or microplate wells.)

One of the major stresses proteins (e.g, antibodies) may encounter is interfacial stress (e.g., from air/water interfaces in liquid formulations, or ice/water interfaces during freezing/thawing.) Surfactants are typically used at or above their CMC concentration(s) to stabilize proteins in biopharmaceutical formulations while under stress or long-term storage to prevent or minimize aggregation and/or particle formation. At or above the surfactant's CMC concentration, the formation of micelles and/or protein-surfactant complexes in aqueous formulation is known to reduce the interfacial stress of the protein, and consequently stabilize proteins against protein-protein interactions and protein particle formation. However, the ability of a surfactant below its CMC concentration to stabilize proteins (e.g., antibodies) in aqueous formulation is relatively unknown, and is often dependent on the antibody, including the antibody's sensitivity to interfacial stress and propensity destabilize to under stress.

Accordingly, “below critical micelle concentration” or “below CMC” is a concentration of surfactant less than which micelles form. In some aspects, the amount of surfactant is at least about 10% below, at least 20% below, at least 30% below, at least 40% below, at least 50% below, at least 60% below, at least 70% below, at least 80% below, at least 90% below, at least 95% below, at least 96% below, at least 97% below, at least 98% below, or at least 99% below the CMC of the surfactant.

Examples of a surfactant include, but are not limited to, anionic surfactants (e.g., ammonium lauryl sulfate, sodium lauryl sulfate, sodium laureth sulfate, sodium myreth sulfate, diocytl sodium sulfosuccinate, perfluorooctanesulfonate, perfluorobutanesulfonate, alkyl-aryl ether phosphates, alkyl ether phosphates, carboxylates, sodium lauroyl sarcosinate, perfluorononanoate, perfluorooctanoate); cationic surfactants (e.g., octenidine dihydrochloride, cetrimonium bromide, cetylpyridinium chloride, benzalkonium chloride, benzethonium chloride, dimethyldioctadecylammonium chloride, and dioctadecyldimethylammonium bromide); zwitterionic (amphoteric) surfactants (e.g., 3-[(3-cholamidopropyl)dimethylammonio]-1-propanesulfonate, cocamidopropyl hydroxysultaine, phosphatidylserine, phosphatidylethanolamine, phosphatidylcholine, sphingomyelins, lauryldimethylamine oxide and myristamine oxide); non-ionic surfactants (e.g., polysorbates or Brij series); ethoxylates (e.g., fatty alcohol ethoxylate (e.g., octaethylene glycol monododecyl ether and pentaethylene glycol monododecyl ether), alkylphenolethoxylates (e.g., nonoxynols and Triton X-100); fatty acid ethoxylates, ethoxylated amines and/or fatty acid amides (e.g., polyethoxylated tallow amine, cocamide monoethanolamine, and cocamide diethanolamine); terminally blocked ethoxylates (e.g., poloxamers); fatty acid esters of polyhydroxy compounds; fatty acid esters of glycerol (e.g., glycerol monostearate and glycerol monolaurate); fatty acid esters of sorbitol (e.g., Spans such as sorbitan monolaurate, sorbitan monostearate, and sorbitan tristearate, and Tweens such as Tween 20, Tween 40, Tween 60, and Tween 80); fatty acid esters of sucrose; alkyl polyglucosides (e.g., decyl glucoside, lauryl glucoside, and octyl glucoside); of a combination thereof.

In some aspects, the surfactant is not polysorbate 20 (PS20).

In some aspects, the surfactant is polysorbate 80 (PS80). PS80 is also known as polyoxyethylene (20) sorbitan monooleate, and is represented by the formula:

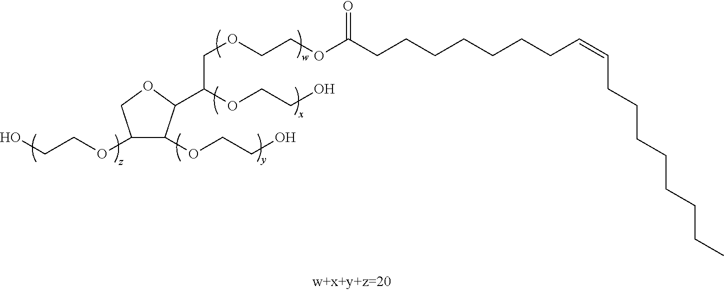

In some aspects, an amount of PS80 below CMC is from about 0.0004% (w/v) to about 0.0012% (w/v), e.g., from about 0.0006% to about 0.0012%, from about 0.0008% to about 0.0012%, from about 0.001% to about 0.0012%, from about 0.0004% to about 0.0010%, from about 0.0006% to about 0.001%, from about 0.0008% to about 0.001%, from about 0.0004% to about 0.0008%, from about 0.0006% to about 0.0008%, or from about 0.0004% to about 0.0006%. In some aspects, an amount of PS80 below CMC is about 0.0004% (w/v), about 0.0006%, about 0.0008%, about 0.001%, or about 0.0012%.

In some aspects, the surfactant is polysorbate 20 (PS20). PS20 is also known as polyoxyethylene (20) sorbitan monolaurate, and is represented by the formula:

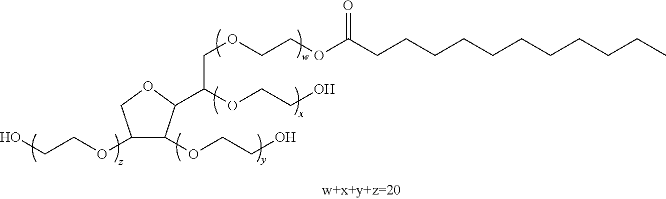

In some aspects, an amount of PS20 below CMC is from about 0.002% (w/v) to about 0.006% (w/v), for example, from about 0.002% to about 0.004% or from about 0.004% to about 0.006%. In some aspects, an amount of PS20 below CMC in the formulations of the present disclosure is about 0.002% (w/v), about 0.004%, or about 0.006%.

In some aspects, the surfactant is a poloxamer. Poloxamers are nonionic triblock copolymers composed of a central hydrophobic chain of polyoxypropylene (poly(propylene oxide)) flanked by two hydrophilic chains of polyoxyethylene (poly(ethylene oxide)). Examples of a poloxamer include, but are not limited to, poloxamer 188, poloxamer 407, poloxamer 184, poloxamer 124, or a combination thereof.

In some aspects, an amount of poloxamer (e.g., poloxamer 188) below CMC is from about 0.03% (w/v) to about 0.08% (w/v), for example, from about 0.03% to about 0.07%, from about 0.03% to about 0.06%, from about 0.03% to about 0.05%, from about 0.03% to about 0.04%, from about 0.04% to about 0.08%, from about 0.04% to about 0.07%, from about 0.04% to about 0.06%, from about 0.04% to about 0.05%, from about 0.05% to about 0.08%, from about 0.05% to about 0.07%, from about 0.05% to about 0.06%, from about 0.06% to about 0.08%, from about 0.06% to about 0.07%, or from about 0.07% to about 0.08%. In some aspects, an amount of a poloxamer (e.g., poloxamer 188) below CMC is from about 0.027% (w/v) to about 0.08% (w/v). In some aspects, an amount of poloxamer (e.g., poloxamer 188) below CMC is about 0.03% (w/v), about 0.04%, about 0.05%, about 0.06%, about 0.07%, or about 0.08%.

In some aspects, the surfactant is a Brij series surfactant (e.g., Brij 35 (dodecyl-poly-ethylene-oxide-ether, CH₃(CH₂)₁₁(OCH₂CH₂)₂₃OH); Brij 93 (polyethylene glycol oleyl ether, polyoxyethylene (2) oleyl ether, C₁₈H₃₅(OCH₂CH₂)ₙOH, n˜2); Brij S100 (polyoxyethylene (100) stearyl ether, C₁₈H₃₇(OCH₂CH₂)ₙOH, n˜100); Brij 58 (polyethylene glycol hexadecyl ether, HO(CH₂CH₂O)₂₀C₁₆H₃₃); Brij C10 (polyethylene glycol hexadecyl ether, polyoxyethylene (10) cetyl ether, C₁₆H₃₃(OCH₂CH₂)ₙOH, n˜10); Brij L4 (polyethylene glycol dodecyl ether, polyoxyethylene (4) lauryl ether, C₂₀H₄₂O₅)ₙ); Brij 020 (polyoxyethylene (20) oleyl ether, C₁₈H₃₅(OCH₂CH₂)ₙOH, n˜20); Brij S10 (polyethylene glycol octadecyl ether, polyoxyethylene (10) stearyl ether, C₁₈H₃₇(OCH₂CH₂)ₙOH, n˜10); or Brij S20 (polyethylene glycol octadecyl ether, polyoxyethylene (20) stearyl ether, C₁₈H₃₇(OCH₂CH₂)ₙOH, n˜20)). In some aspects, an amount of a Brij series surfactant below CMC is from about 0.001% (w/v) to about 0.01%, for example, from about 0.001% to about 0.009%, from about 0.001% to about 0.007%, from about 0.001% to about 0.005%, from about 0.001% to about 0.003%, from about 0.003% to about 0.01%, from about 0.003% to about 0.009%, from about 0.003% to about 0.007%, from about 0.003% to about 0.005%, from about 0.005% to about 0.01%, from about 0.005% to about 0.009%, from about 0.005% to about 0.007%, from about 0.007% to about 0.01%, or from about 0.007% to about 0.009%. In some aspects, an amount of a Brij series surfactant below CMC is about 0.003% (w/v) or about 0.0089% (w/v).

In some aspects, the surfactant is tocopheryl polyethylene glycol succinate (TPGS). In some aspects, the TPGS is vitamin E TPGS 1000. In some aspects, an amount of TPGS below CMC is from about 0.001% (w/v) to about 0.02%, for example, from about 0.001% to about 0.01%, from about 0.001% to about 0.007%, from about 0.001% to about 0.005%, from about 0.001% to about 0.003%, from about 0.003% to about 0.02%, from about 0.003% to about 0.01%, from about 0.003% to about 0.007%, from about 0.003% to about 0.005%, from about 0.005% to about 0.02%, from about 0.005% to about 0.01%, from about 0.005% to about 0.007%, from about 0.007% to about 0.02%, from about 0.007% to about 0.01%, or from about 0.01% to about 0.02%. In some aspects, an amount of TPGS below CMC is about 0.0054% (w/v) or about 0.0162% (w/v).

In some aspects, anti-IL5R antibody formulations of the present disclosure can contain various other components. In some aspects, the formulations comprise a buffer (e.g., histidine, acetate, phosphate or citrate buffer) and/or a stabilizer agent (e.g. human albumin), etc., or a combination thereof. In some aspects, the formulations comprise one or more pharmaceutically acceptable carriers, including, e.g., ion exchangers, alumina, aluminum stearate, lecithin, serum proteins, such as human serum albumin, buffer substances such as phosphates, sucrose, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, polyethylene-polyoxypropylene-block polymers, and polyethylene glycol, or a combination thereof.

In some aspects, anti-IL5R antibody formulations of the present disclosure comprise histidine. In some aspects, the formulations comprise from about 1 mM to about 100 mM histidine, for example, from about 5 mM to about 80 mM histidine, from about 10 mM to about 60 mM histidine, from about 15 mM to about 50 mM histidine, from about 15 mM to about 30 mM histidine, or about 20 mM histidine.

In some aspects, anti-IL5R antibody formulations of the present disclosure comprise an uncharged excipient. The term “excipient” refers to a pharmacologically inactive substance formulated with an antibody or antigen binding fragment thereof as described herein. In some aspects, the uncharged excipient can assist in the prevention of denaturation or otherwise assist in stabilizing the antibody or antigen binding fragment thereof. Examples of excipients are known in the art. Examples can be taken, e.g., from the handbook: Gennaro, Alfonso R.: “Remington's Pharmaceutical Sciences”, Mack Publishing Company, Easton, Pa., 1990. In some aspects, the uncharged excipient is fructose, glucose, mannose, sorbose, xylose, lactose, maltose, sucrose, dextran, pullulan, dextrin, cyclodextrins, soluble starch, trehalose, sorbitol, erythritol, isomalt, lactitol, maltitol, xylitol, glycerol, lactitol, hydroxyethyl starch, water-soluble glucans, or a combination thereof. In some aspects, the uncharged excipient is sucrose. In some aspects, the uncharged excipient is trehaolse. In some aspects, the uncharged excipient is mannitol.

In some aspects, the uncharged excipient is present in anti-IL5R antibody formulations in an amount of from about 1 mM to about 1 M, about 2 mM to about 500 mM, about 5 mM to about 400 mM, about 10 mM to about 300 mM, or about 20 mM to about 250 mM. In some aspects, the uncharged excipient is from about 5 mM to about 150 mM, about 10 mM to about 100 mM, about 20 mM to about 80 mM, about 30 mM, about 40 mM, about 50 mM, about 60 mM, or about 70 mM in the formulation. In one aspect, the uncharged excipient is about 50 mM in the formulation. In some aspects, the uncharged excipient is about 50 mM to about 800 mM, about 100 mM to about 500 mM, about 150 mM to about 400 mM, about 200 mM, about 400 mM, about 200 mM, about 300 mM, or about 250 mM in the formulation. In one aspect, the uncharged excipient is about 250 mM in the formulation.

In other aspects, the unchanged excipient is from about 0.5% (w/v) to about 8.5% (w/v) in the formulation, for example, from about 0.5% to about 8%, from about 0.5% to about 6%, from about 0.5% to about 4%, from about 0.5% to about 2%, from about 0.5% to about 1%, from about 1% to about 8.5%, from about 1% to about 8%, from about 1% to about 6%, from about 1% to about 4%, from about 1% to about 2%, from about 2% to about 8.5%, from about 2% to about 8%, from about 2% to about 6%, from about 2% to about 4%, from about 4% to about 8.5%, from about 4% to about 8%, from about 4% to about 6%, from about 6% to about 8%, or from about 6% to about 8.5%.

In some aspects, the uncharged excipient is trehalose, as represented by the formula:

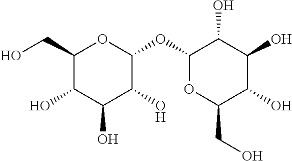 [SMILES: OC[C@H]1O[C@H](O[C@H]2O[C@H](CO)[C@@H](O)[C@H](O)[C@H]2O)[C@H](O)[C@@H](O)[C@@H]1O]

In some aspects, the trehalose is about 1 mM to about 1 M, about 2 mM to about 500 mM, about 5 mM to about 400 mM, about 10 mM to about 300 mM, or about 20 mM to about 250 mM in the formulation. In some aspects, the trehalose is about 5 mM to about 150 mM, about 10 mM to about 100 mM, about 20 mM to about 80 mM, about 30 mM, about 40 mM, about 50 mM, about 60 mM, or about 70 mM in the formulation. In one aspect, the trehalose is about 50 mM in the formulation. In some aspects, the trehalose is about 50 mM to about 800 mM, about 100 mM to about 500 mM, about 150 mM to about 400 mM, about 200 mM, about 400 mM, about 200 mM, about 300 mM, or about 250 mM in the formulation. In one aspect, the trehalose is about 250 mM in the formulation.

In some aspects, anti-IL5R antibody formulations of the present disclosure comprise arginine. Arginine is a conditionally non-essential amino acid that can be represented by the formula:

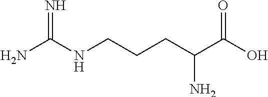 [SMILES: N=C(N)NCCCC(N)C(=O)O]

Arginine, as used herein, includes the free base form of arginine, as well as any and all salts thereof. In some aspects, arginine includes a pharmaceutically acceptable salt thereof, e.g., arginine hydrochloride. Arginine, as used herein, also includes all enantiomers (e.g., L-arginine and S-arginine), and any combination of enantiomers (e.g., 50% L-arginine and 50% S-arginine; 90%-100% L-arginine and 10%-0% S-arginine, etc.). In some aspects, the term “arginine” includes greater than 99% L-arginine and less than 1% S-arginine. In some aspects, the term “arginine” includes an enantomerically pure L-arginine. In some aspects, arginine is a pharmaceutical grade arginine.

Various concentrations of arginine can be present in the anti-IL5R antibody formulations of the present disclosure. In some aspects, the formulation comprises greater than 50 mM arginine, greater than 75 mM arginine, greater than 100 mM arginine, greater than 125 mM arginine, greater than 130 mM arginine, greater than 150 mM arginine, greater than 175 mM arginine, or greater than 200 mM arginine. In other aspects, the formulation comprises up to 800 mM arginine, up to 600 mM arginine, up to 400 mM arginine, up to 200 mM arginine, up to 150 mM arginine, up to 130 mM arginine, or up to 125 mM arginine. In other aspects, the formulation comprises 50 mM to 300 mM, 75 mM to 250 mM, 100 mM to 200 mM, 110 mM to 160 mM, 120 mM to 150 mM, or about 125 mM arginine. In some aspects, the formulation comprises 125 mM arginine. In some aspects, the formulation comprises 130 mM arginine. In some aspects, arginine is added in an amount sufficient to maintain osmolality of the formulation. In some aspects, arginine is added in an amount sufficient to achieve a hyper-tonic solution.

Various buffers can be present in the anti-IL5R antibody formulations of the present disclosure. In some aspects, the buffer is acetate. In some aspects, the buffer is succinate.

Anti-IL5R antibody formulations of the present disclosure can have various viscosities. Methods of measuring viscosity of antibody formulations are known to those in the art, and can include, e.g., a rheometer (e.g., Anton Paar MCR301 Rheometer with either a 50 mm, 40 mm or 20 mm plate accessory). In some aspects, the formulation has a viscosity of less than 20 centipoise (cP), less than 18 cP, less than 15 cP, less than 13 cP, or less than 11 cP. In some aspects, the formulation has a viscosity of less than 13 cP. One of skill in the art will appreciate that viscosity is dependent on temperature, thus, unless otherwise specified, the viscosities provided herein are measured at 25° C. unless otherwise specified.

Anti-IL5R antibody formulations of the present disclosure can have different osmolarity concentrations. Methods of measuring osmolarity of antibody formulations are known in the art, and can include, e.g., an osmometer (e.g., an Advanced Instrument Inc. 2020 freezing point depression osmometer). In some aspects, the formulation has an osmolarity of between 200 and 600 mosm/kg, between 260 and 500 mosm/kg, or between 300 and 450 mosm/kg.

Anti-IL5R antibody formulations of the present disclosure can have various pH levels. In some aspects, the pH of the formulation is between 4 and 7, between 4.5 and 6.5, or between 5 and 6. In some aspects, the pH of the formulation is 5. In some aspects, the pH of the formulation is 6. In some aspects, the pH of the formulation is >7. Various means may be utilized in achieving the desired pH level, including, but not limited to the addition of the appropriate buffer. In some aspects, the pH of the formulation is about 5.5 to about 6.0. In some aspects, the pH of the formulation is about 5.5. In some aspects, the pH of the formulation is about 5.6.

In some aspects, various components can be omitted from anti-IL5R antibody formulations, or can be “substantially free” of that component. The term “substantially free” as used herein refers to a formulation containing less than 0.01%, less than 0.001%, less than 0.0005%, less than 0.0003%, or less than 0.0001% of the designated component.

In some aspects, anti-IL5R antibody formulations of the present disclosure are substantially free of a saccharide, i.e., contains less than 0.01% (w/v), less than 0.001%, less than 0.0005%, less than 0.0003%, or less than 0.0001% of a saccharide. The term “saccharide” as used herein refers to a class of molecules that are derivatives of polyhydric alcohols. Saccharides are commonly referred to as carbohydrates and may contain different amounts of sugar (saccharide) units, e.g., monosaccharides, disaccharides and polysaccharides. In some aspects, the formulation is substantially free of disaccharide. In some aspects, the formulation is substantially free of a reducing sugar, a non-reducing sugar, or a sugar alcohol. In some aspects, the formulation is substantially free of proline, glutamate, sorbitol, divalent metal ions, and/or succinate.

In some aspects, an anti-IL5R antibody formulation of the present disclosure comprises (i) about 2 mg/mL to about 100 mg/mL of an anti-IL5R antibody or antibody binding fragment thereof (e.g., comprising a heavy chain variable region CDR1 comprising the sequence set forth in SEQ ID NO: 1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6); and (ii) an amount of surfactant below CMC (e.g., about 0.0004% (w/v) to about 0.0012% (w/v) of PS80, or about 0.03% (w/v) to about 0.08% (w/v) poloxamer (e.g., poloxamer 188)). In some aspects, the formulation further comprises an uncharged excipient, arginine, and/or histidine. In some aspects, the formulation further comprises (iii) about 1.5% (w/v) to about 8.5% (w/v) of an uncharged excipient (e.g., trehalose); (iv) about 100 mM to about 200 mM arginine (e.g., L-arginine); and (v) about 15 mM to about 30 mM histidine.

In some aspects, an anti-IL5R antibody formulation of the present disclosure comprises (i) anti-IL5R antibody or antibody binding fragment thereof, (ii) PS80, (iii) trehalose, (iv) arginine, and (v) histidine. In some aspects, the formulation comprises (i) about 2 mg/mL to about 100 mg/mL of an anti-IL5R antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0004% (w/v) PS80; (iii) about 250 mM trehalose; (iv) about 130 mM L-arginine; and (v) about 20 mM histidine.

In some aspects, the formulation comprises (i) about 2 mg/mL to about 100 mg/mL of an anti-IL5R antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0012% (w/v) PS80; (iii) about 250 mM trehalose; (iv) about 130 mM L-arginine; and (v) about 20 mM histidine.

In some aspects, an anti-IL5R antibody formulation of the present disclosure comprises (i) anti-IL5R antibody or antibody binding fragment thereof, (ii) poloxamer, (iii) trehalose, (iv) arginine, and (v) histidine. In some aspects, the formulation comprises (i) about 2 mg/mL to about 100 mg/mL of an anti-IL5R antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.03% (w/v) poloxamer; (iii) about 250 mM trehalose; (iv) about 130 mM L-arginine; and (v) about 20 mM histidine.

In some aspects, the formulation comprises (i) about 2 mg/mL to about 100 mg/mL of an antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.08% (w/v) poloxamer; (iii) about 250 mM trehalose; (iv) about 130 mM L-arginine; and (v) about 20 mM histidine.

In some aspects, the formulation comprises (i) about 30 mg/mL of an antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0004% (w/v) PS80; (iii) about 250 mM trehalose; (iv) about 130 mM L-arginine; and (v) about 20 mM histidine.

In some aspects, the formulation comprises (i) about 30 mg/mL of an antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.0012% (w/v) PS80; (iii) about 250 mM trehalose; (iv) about 130 mM L-arginine; and (v) about 20 mM histidine.

In some aspects, the formulation comprises (i) about 30 mg/mL of an antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.03% (w/v) poloxamer 188; (iii) about 250 mM trehalose; (iv) about 130 mM L-arginine; and (v) about 20 mM histidine.

In some aspects, the formulation comprises (i) about 30 mg/mL of an antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO: 5; and a light chain variable region CDR3 comprising the sequence set forth in SEQ ID NO:6; (ii) about 0.08% (w/v) poloxamer 188; (iii) about 250 mM trehalose; (iv) about 130 mM L-arginine; and (v) about 20 mM histidine.

In some aspects, the formulation, comprises: (i) about 2 mg/mL to about 100 mg/mL (e.g., about 30 mg/mL) of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO: 3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer; (iii) about 250 mM sucrose or about 250 mM mannitol; and (iv) acetate or succinate.

In some aspects, an anti-IL5R antibody formulation of the present disclosure is suitable for intravenous, subcutaneous, or intramuscular administration.

In some aspects, an anti-IL5R antibody formulation of the present disclosure is a liquid formulation. In some aspects, an anti-IL5R antibody formulation of the present disclosure is an aqueous formulation. In other aspects, the formulation is a lyophilized formulation.

In some aspects, an anti-IL5R antibody formulation of the present disclosure can be sterilized. Antibody formulations can be sterilized by various sterilization methods, including sterile filtration, radiation, etc. In one aspect, an anti-IL5R antibody formulation of the present disclosure is filter-sterilized with a presterilized 0.2 micron filter.

Other aspects of the present disclosure are directed to a dosage form comprising an anti-IL5R antibody formulation provided herein in a container. In some aspects, the container is a syringe. In some aspects, the syringe (e.g., prefilled syringe) comprises a formulation comprising an anti-IL5R antibody or antigen binding fragment thereof and a surfactant at an amount below CMC of the surfactant. In some aspects, the syringe (e.g., prefilled syringe) comprises a formulation comprising an anti-IL5R antibody or antigen binding fragment thereof, PS80 or poloxamer, trehalose, arginine, and histidine. In some aspects, the formulation comprises (i) about 2 mg/mL to about 100 mg/mL of an antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; and a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and (ii) about 0.0004% (w/v) to about 0.0012% (w/v) PS80, or about 0.03% (w/v) to about 0.08% (w/v) poloxamer. In some aspects, the formulation further comprises (iii) about 1.5% (w/v) to about 8.5% (w/v) trehalose; (iv) about 100 mM to about 200 mM L-arginine; and (v) about 15 mM to about 30 mM histidine. In some aspects, the formulation, comprises: (i) about 2 mg/mL to about 100 mg/mL (e.g., about 30 mg/mL) of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer; (iii) about 250 mM sucrose or about 250 mM mannitol; and (iv) acetate or succinate. In some aspects, the syringe (e.g., prefilled syringe) contains about 1 ml, about 2 ml, about 3 ml, about 4 ml, about 5 ml, about 6 ml, about 7 ml, about 8 ml, about 9 ml, about 10 ml, about 15 ml, or about 20 ml a formulation provided herein.

In some aspects, the syringe can be filled with anti-IL5R antibody formulation immediately prior to administration to a subject, e.g., less than 1 week, 1 day, 6 hours, 3 hours, 2 hours, 1 hour, 30 minutes, 20 minutes, or 10 minutes prior to administration to a subject. In some aspects, the syringe is filled with the anti-IL5R antibody formulation at the point of retail sale, or by the facility for which treatment of the subject occurs. In some aspects, the syringe is pre-filled, e.g., the syringe is filled with the formulation greater than 1 day, 2 days, 4 days, 1 week, 2 weeks, 1 month, 2 months, 3 months, 6 months, 12 months, 18 months, 24 months, 3 years, or 4 years prior to administration to a subject. In some aspects, the syringe (e.g., prefilled syringe) comprises a needle, e.g., a 27 G regular wall needle, a 27 G thin wall needle, a 29 G regular wall needle, or a 29 G thin wall needle. In some aspects, the syringe (e.g., prefilled syringe) comprises a 29 G thin wall needle.

In some aspects, the syringe is a plastic syringe or a glass syringe. In some aspects, the syringe is made of materials that are substantially free from tungsten. In some aspects, the syringe is coated with silicone. In some aspects, the syringe comprises a plunger having a fluoropolymer resin disk. Examples of syringes can include, but are not limited to Hypak™ for Biotech 1 ml Long (Becton Dickinson), with a Becton Dickinson Hypak 1 mL long plunger stopper 4023 Flurotec Daikyo SilOOO (Catalog #47271919), C3Pin (lot #E912701), Hypak™ for Biotech 0.8 mg silicone oil (Becton Dickinson), and CZ syringes (West, Catalog #19550807).

Other aspects of the present disclosure are directed to a vial comprising an anti-IL5R antibody formulation described herein. In some aspects, the vial is a plastic vial or glass vial. In some aspects, the vial comprises a formulation comprising an anti-IL5R antibody or antigen binding fragment thereof and a surfactant at an amount below CMC of the surfactant. In some aspects, the vial comprises a formulation comprising an anti-IL5R antibody or antigen binding fragment thereof, PS80 or poloxamer, trehalose, arginine, and histidine. In some aspects, the formulation comprises (i) about 2 mg/mL to about 100 mg/mL of an antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and (ii) about 0.0004% (w/v) to about 0.0012% (w/v) PS80, or about 0.03% (w/v) to about 0.08% (w/v) poloxamer. In some aspects, the formulation further comprises (iii) about 1.5% (w/v) to about 8.5% (w/v) trehalose; (iv) about 100 mM to about 200 mM L-arginine; and (v) about 15 mM to about 30 mM histidine. In some aspects, the formulation, comprises: (i) about 2 mg/mL to about 100 mg/mL (e.g., about 30 mg/mL) of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer; (iii) about 250 mM sucrose or about 250 mM mannitol; and (iv) acetate or succinate. In some aspects, the vial contains about 1 ml, about 2 ml, about 3 ml, about 4 ml, about 5 ml, about 6 ml, about 7 ml, about 8 ml, about 9 ml, about 10 ml, about 15 ml, or about 20 ml a formulation described herein.

Other aspects of the present disclosure are directed to a kit comprising an anti-IL5R antibody formulation, dosage form, vial or syringe (e.g., prefilled syringe) described herein, and instructions for use. In some aspects, the kit comprises a formulation comprising an anti-IL5R antibody or antigen binding fragment thereof and a surfactant at an amount below CMC of the surfactant. In some aspects, the kit comprises a formulation comprising an anti-IL5R antibody or antigen binding fragment thereof, PS80 or poloxamer, trehalose, arginine, and histidine. In some aspects, the formulation comprises (i) about 2 mg/mL to about 100 mg/mL of an antibody or antibody binding fragment thereof comprising a heavy chain variable region CDR1 comprising the sequence set forth in SEQ ID NO:1; a heavy chain variable region CDR2 comprising the sequence set forth in SEQ ID NO:2; and a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and (ii) about 0.0004% (w/v) to about 0.0012% (w/v) PS80, or about 0.03% (w/v) to about 0.08% (w/v) poloxamer. In some aspects, the formulation further comprises (iii) about 1.5% (w/v) to about 8.5% (w/v) trehalose; (iv) about 100 mM to about 200 mM L-arginine; and (v) about 15 mM to about 30 mM histidine. In some aspects, the formulation, comprises: (i) about 2 mg/mL to about 100 mg/mL (e.g., about 30 mg/mL) of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; (ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer; (iii) about 250 mM sucrose or about 250 mM mannitol; and (iv) acetate or succinate.

In some aspects, anti-IL5R antibody formulations of the present disclosure are “stable” and/or have “improved stability.” As used herein, the terms “stable” and “stability” generally relate to maintaining the integrity or to minimizing the degradation, denaturation, aggregation or unfolding of a biologically active agent such as a protein, peptide or another bioactive macromolecule. As used herein, “improved stability” generally means that, under conditions known to result in degradation, denaturation, aggregation or unfolding, the protein (e.g., anti-IL5R antibody), peptide or another bioactive macromolecule of interest maintains greater stability compared to a control protein, peptide or another bioactive macromolecule.

In some aspects, stability is determined by particle formation (e.g., subvisible particle formation) and/or fragmentation of anti-IL5R antibody. In some aspects, a subvisible particle has a diameter of ≥1 μm, ≥2 μm, ≥5 μm, ≥10 μm, or ≥25 μm.

The term “stable” can be relative and not absolute. Thus, in some aspects, an anti-IL5R antibody is stable if less than 20%, less than 15%, less than 10%, less than 5%, or less than 2% of the antibody is degraded, denatured, aggregated, or unfolded, as determined by size exclusion chromatography (SEC) high performance liquid chromatography (HPLC) when the antibody is stored at 2° C. to 8° C. for 6 months. In some aspects, the antibody is stable if less than 20%, less than 15%, less than 10%, less than 5%, or less than 2% of the antibody is degraded, denatured, aggregated, or unfolded, as determined by SEC HPLC when the antibody is stored at 2° C. to 8° C. for 12 months. In some aspects, the antibody is stable if less than 20%, less than 15%, less than 10%, less than 5% or less than 2% of the antibody is degraded, denatured, aggregated, or unfolded, as determined by SEC HPLC when the antibody is stored at 2° C. to 8° C. for 18 months. In some aspects, the antibody is stable if less than 20%, less than 15%, less than 10%, less than 5%, or less than 2% of the antibody is degraded, denatured, aggregated, or unfolded, as determined by SEC HPLC when the antibody is stored at 2° C. to 8° C. for 24 months.

In some aspects, the antibody is stable if less than 20%, less than 15%, less than 10%, less than 5%, or less than 2% of the antibody is degraded, denatured, aggregated, or unfolded, as determined by SEC HPLC when the antibody is stored at 23° C. to 27° C. for 3 months. In some aspects, the antibody is stable if less than 20%, less than 15%, less than 10%, less than 5% or less than 2% of the antibody is degraded, denatured, aggregated, or unfolded, as determined by SEC HPLC when the antibody is stored at 23° C. to 27° C. for 6 months. In some aspects, the antibody is stable if less than 20%, less than 15%, less than 10%, less than 5%, or less than 2% of the antibody is degraded, denatured, aggregated, or unfolded, as determined by SEC HPLC when the antibody is stored at 23° C. to 27° C. for 12 months. In some aspects, the antibody is stable if less than 20%, less than 15%, less than 10%, less than 5%, or less than 2% of the antibody is degraded, denatured, aggregated, or unfolded, as determined by SEC HPLC when the antibody is stored at 23° C. to 27° C. for 24 months.

In some aspects, the antibody is stable if less than 6%, less than 4%, less than 3%, less than 2%, or less than 1% of the antibody is degraded, denatured, aggregated, or unfolded per month, as determined by SEC HPLC when the antibody is stored at 40° C. In some aspects, the antibody is stable if less than 6%, less than 4%, less than 3%, less than 2%, or less than 1% of the antibody is degraded, denatured, aggregated, or unfolded per month, as determined by SEC HPLC when the antibody is stored at 5° C.

In some aspects, the antibody formulations of the present invention can be considered stable if the antibody exhibits very little to no loss of the binding activity of the antibody (including antibody fragments thereof) of the formulation compared to a reference antibody as measured by antibody binding assays known to those in the art, such as, e.g., enzyme-linked immunosorbent assays (ELISAs), etc., over a period of 8 weeks, 4 months, 6 months, 9 months, 12 months, or 24 months. In some aspects, the antibody stored at about 40° C. for at least 1 month retains at least 80%, at least about 85%, at least about 90%, at least about 95%, at least about 98%, or at least about 99% of binding ability to an IL-5 receptor polypeptide compared to a reference antibody which has not been stored. In some aspects, the antibody stored at about 5° C. for at least 6 months retains at least 80%, at least about 85%, at least about 90%, at least about 95%, at least about 98%, or at least about 99% of binding ability to an IL-5 receptor polypeptide compared to a reference antibody which has not been stored. In some aspects, the antibody stored at about 40° C. for at least 1 month retains at least 95% of binding ability to an IL-5 receptor polypeptide compared to a reference antibody which has not been stored. In some aspects, the antibody stored at about 5° C. for at least 6 months retains at least 95% of binding ability to an IL-5 receptor polypeptide compared to a reference antibody which has not been stored.

In some aspects, an anti-IL5R antibody formulation of the present disclosure has less than about 20,000 subvisible particles per mL, less than about 10,000 subvisible particles per mL, less than about 5,000 subvisible particles per mL, less than about 1,000 subvisible particles per mL, less than about 500 subvisible particles per mL, less than about 100 subvisible particles per mL, less than about 50 subvisible particles per mL, or less than about 20 subvisible particles per mL. In some aspects, the formulation has been subjected to aggressive agitation (e.g., for about 10 days, e.g., at room temperature), freeze-thaw cycles (e.g., from about 1 to about 10 cycles), and/or storage (e.g., for about 1 month, e.g., at about 40° C.).

In other aspects, an anti-IL5R antibody formulation of the present disclosure has about 20% less, about 30% less, about 40% less, about 50% less, about 60% less, about 70% less, about 80% less, about 90% less, about 95% less, or about 99% less subvisible particles than a control formulation.

In some aspects, an anti-IL5R antibody formulation of the present disclosure is essentially free of particles. As used herein, the term “essentially free of particles” refers to the absence of visible particles when viewed under a light box. In some aspects, essentially free of particles refers to a formulation containing less than about 200 particles/mL, less than about 150 particles/mL, less than about 100 particles/mL, less than about 50 particles/mL, less than about 30 particles/mL, less than about 20 particles/mL, less than about 15 particles/mL, less than about 10 particles/mL, less than about 5 particles/mL, less than about 2 particles/mL or less than about 1 particle/mL. In some aspects, essentially free of particles refers to formulations containing about 1 to about 20 particles/mL, about 10 to about 150 particles/mL, about 1 to about 50 particles/mL, about 2 to about 40 particles/mL, about 3 to about 30 particles/mL, about 4 to about 25 particles/mL, or about 5 to about 20 particles/mL.

In some aspects, particles are detected by visual inspection, micro-flow imaging (MFI), size-exclusion chromatography (SEC), and/or a particle counter (e.g., IAC particle counter). In some aspects, protein fragmentation is detected by gel electrophoresis.

### II. Methods of Use

In some aspects, the present disclosure is directed to methods of treating or preventing a disease or disorder characterized by aberrant expression and/or activity of an IL-5 polypeptide, a disease or disorder characterized by aberrant expression and/or activity of an IL-5 receptor (IL5R) or one or more subunits thereof, an autoimmune disease, an inflammatory disease, a proliferative disease, or an infection (preferably, a respiratory infection), or one or more symptoms thereof, comprising administering an anti-IL5R antibody formulation, dosage form, vial and/or syringe (e.g., prefilled syringe) provided herein.

In some aspects, the present disclosure is directed to methods of treating or preventing a pulmonary disease or disorder comprising administering an anti-IL5R antibody formulation, dosage form, vial and/or syringe (e.g., prefilled syringe) provided herein.

In some aspects, the pulmonary disease or disorder is an eosinophilic disease or disorder. In some aspects, the pulmonary disease or disorder is asthma, chronic obstructive pulmonary disease (COPD), allergic bronchopulmonary aspergillosis, acute and chronic eosinophilic bronchitis, acute and chronic eosinophilic pneumonia, Churg-Strauss syndrome, hypereosinophilic syndrome, drug, irritant and radiation-induced pulmonary eosinophilia, infection-induced pulmonary eosinophilia, autoimmune-related pulmonary eosinophilia, eosinophilic esophagitis, Crohn's disease, or a combination thereof. In some aspects, the asthma is eosinophilic asthma, neutrophilic asthma, combined eosinophilic and neutrophilic asthma, or aspirin sensitive asthma.

The terms “treat” and “treatment” refer to both therapeutic treatment and prophylactic, maintenance, or preventative measures, wherein the object is to prevent or alleviate (lessen) an undesired physiological condition, disorder or disease, or obtain beneficial or desired clinical results. The terms “treat,” “treatment,” and “treating” refer to the reduction or amelioration of the progression, severity, and/or duration of such a disease or disorder (e.g., a disease or disorder characterized by aberrant expression and/or activity of an IL-5 polypeptide, a disease or disorder characterized by aberrant expression and/or activity of an IL-5 polypeptide or one or more subunits thereof, an autoimmune disease, an inflammatory disease, a proliferative disease, or an infection) or the amelioration of one or more symptoms thereof resulting from the administration of one or more therapies (including, but not limited to, the administration of one or more prophylactic or therapeutic agents). In some aspects, such terms refer to reduction in inflammation associated eosinophil-mediated inflammation. In other aspects, such terms refer to the reduction of the release of inflammatory agents by mast cells, or the reduction of the biological effect of such inflammatory agents. In other aspects, such terms refer to a reduction of the growth, formation and/or increase in the number of hyperproliferative cells (e.g., cancerous cells). In yet other aspects, such terms refer to the reduction of inflammation of the airways, skin, gastrointestinal tract, or combinations thereof. In yet other aspects, such terms refer to the reduction in the symptoms associated with asthma. In some aspects, such terms refer to the reduction in the symptoms associate with chronic obstructive pulmonary disease (COPD).

As used herein, the term “therapeutically effective amount” refers to the amount of therapy (e.g., anti-IL5R antibody or antigen binding fragment thereof), that is sufficient to reduce the severity of a disease or disorder or one or more symptoms thereof, reduce the duration of a respiratory condition, ameliorate one or more symptoms of such a disease or disorder, prevent the advancement of such a disease or disorder, cause regression of such a disease or disorder, or enhance or improve the therapeutic effect(s) of another therapy. In some aspects, the therapeutically effective amount cannot be specified in advance and can be determined by a caregiver, for example, by a physician or other healthcare provider, using various means, for example, dose titration. Appropriate therapeutically effective amounts can also be determined by routine experimentation using, for example, animal models.

The formulations as provided herein can be suitable for treatment of a subject. As used herein, “subject” can be used interchangeably with “patient” and refers to any animal classified as a mammal, including humans and non-humans, such as, but not limited to, domestic and farm animals, zoo animals, sports animals, and pets. In some aspects, subject refers to a human.

The route of administration of the anti-IL5R antibody formulations of the present disclosure can be via, for example, oral, parenteral, inhalation or topical modes of administration. The term parenteral as used herein includes, e.g., intravenous, intraarterial, intraperitoneal, intramuscular, subcutaneous, rectal or vaginal administration. In some aspects, anti-IL5R antibody formulations of the present disclosure are suitable for administration via injection, in particular, for intravenous or intraarterial injection, or drip.

### EXAMPLES

### Example 1

Studies were performed to test the effects of different stressors on the development of subvisible particles in anti-IL5R antibody formulations containing different surfactants, particularly, polysorbate 20 (PS20), polysorbate 80 (PS80), or poloxamer 188 at an amount below the critical micelle concentration (CMC) of the surfactant.

Formulations were prepared containing 30 mg/ml anti-IL5R antibody, 20 mM histidine/histidine hydrochloride (HCl), and 250 mM trehalose dihydrate, pH 6.0 with either no surfactant or 0.006% polysorbate 20 (PS20), 0.02% PS20, 0.0004% polysorbate 80 (PS80), 0.0012% PS80, 0.006% PS80, 0.02% PS80, 0.027% poloxamer 188, or 0.08% poloxamer 188. Anti-IL5R antibody was manufactured at DSC, MEDI-563 lot MS00684-42, PFB at 134 g/L. Using the source antibody, samples were reformulated by dilution and buffer exchange to achieve the desired concentrations. Surfactants were spiked from a liquid stock with a higher concentration. Final formulations were sterile filtered with 0.2 um filters.

For stability testing, 1 mL of the formulations was added to a syringe (Becton Dickinson Hypak 1 mL syringe, 29-gauge needle, with West 4023 plunger stopper) or glass vial (SCHOTT 2R type I glass vial, with West Daikyo 13 mm stopper) using manual filling and stoppering.

Aggressive agitation (end-to-end rotation) was then performed with a Scientific Industries Genie SI-1100 Roto-Shake Rotator/Rocker at the speed of 35 rpm for a total of 10 days. After agitation, syringes were emptied through the needle and formulation samples from syringes and glass vials were tested with a Micro Flow Imaging (MFI) instrument. For testing, 0.2 mL of each sample was first purged, followed by analysis of 0.5 mL of each sample. Subvisible particles (≥22 μm and ≥25 μm) were measured at 5 and 10 days by the total count of pictures captured by the instrument. Aspect ratio filtration was applied to avoid counting air bubbles for better accuracy.

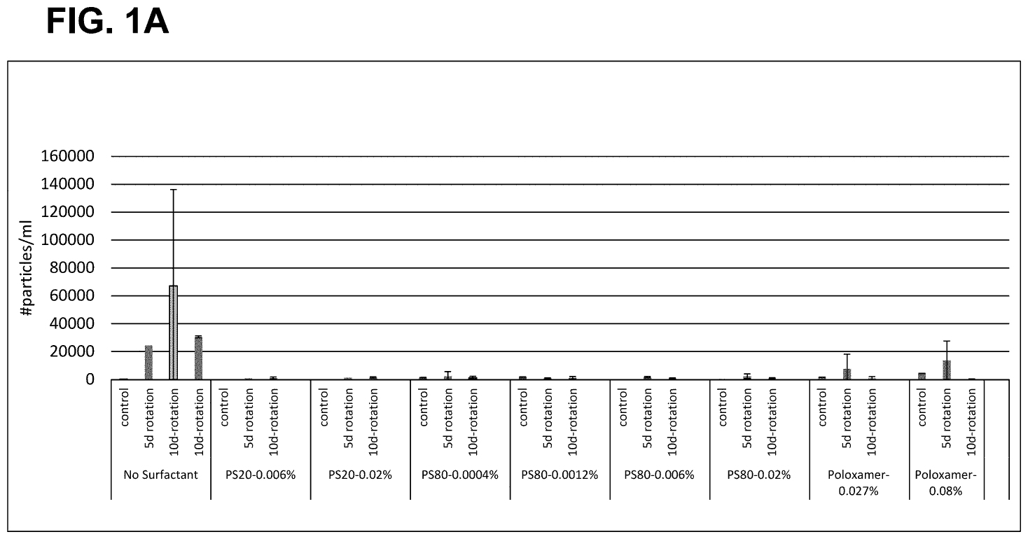

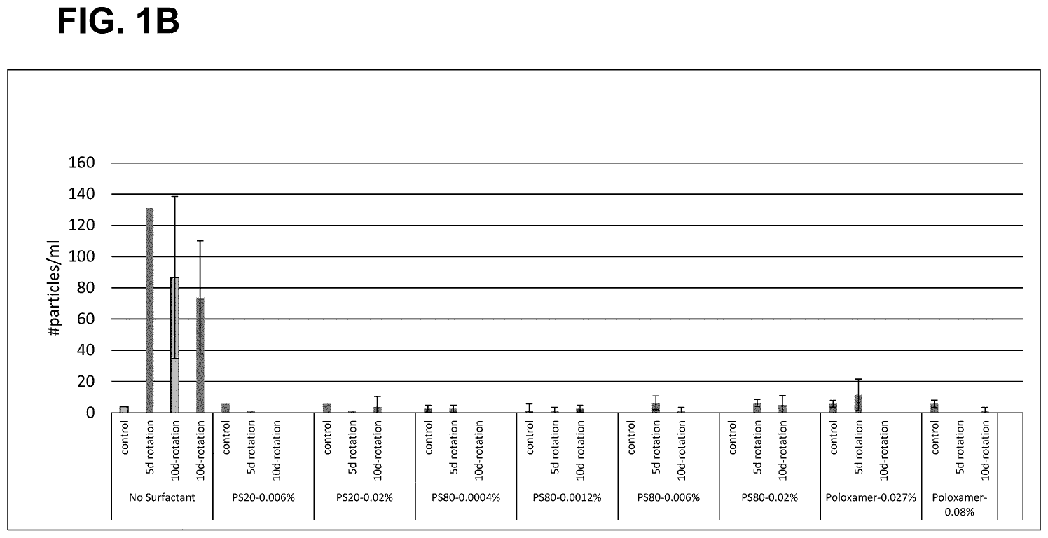

FIGS. 1A-1B show that formulations in prefilled syringes containing no surfactant had a significant presence of subvisible particles (≥2 μm and ≥25 μm, respectively) after 5 days and 10 days of agitation. As expected, formulations in prefilled syringes containing an amount of surfactant above CMC (0.02% PS20, 0.006% PS80, and 0.02% PS80) did not have a significant presence of subvisible particles after 10 days of rotation. However, formulations in prefilled syringes containing an amount of surfactant below CMC (0.006% PS20, 0.0004% PS80, 0.0012% PS80, 0.027% poloxamer 188, and 0.08% poloxamer 188) also did not have a significant presence of subvisible particles after 10 days of rotation.

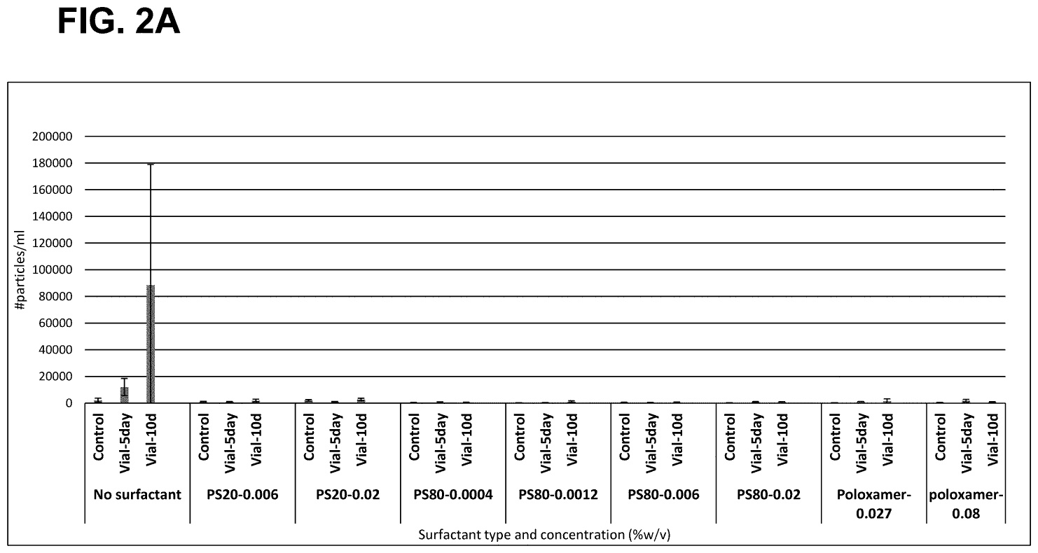

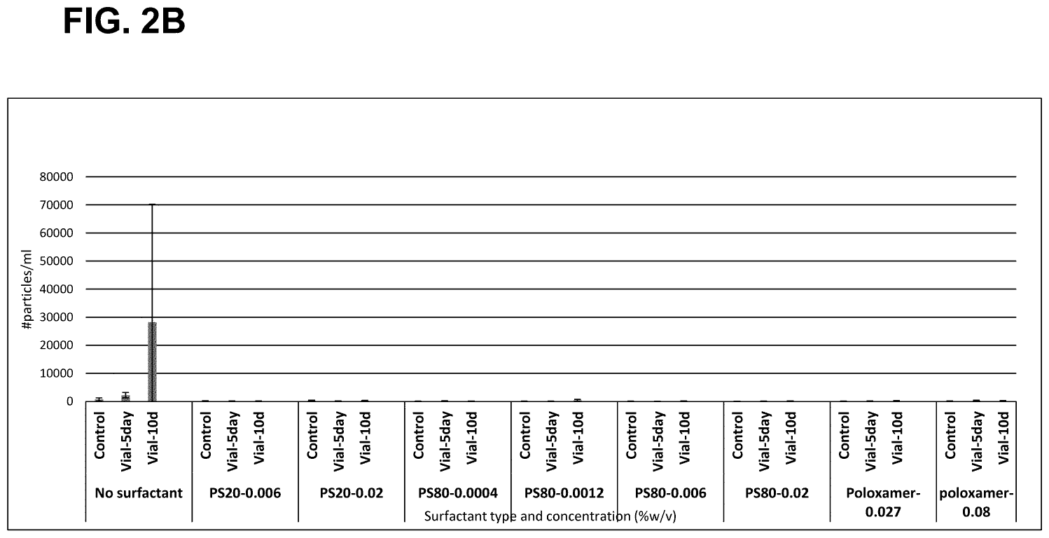

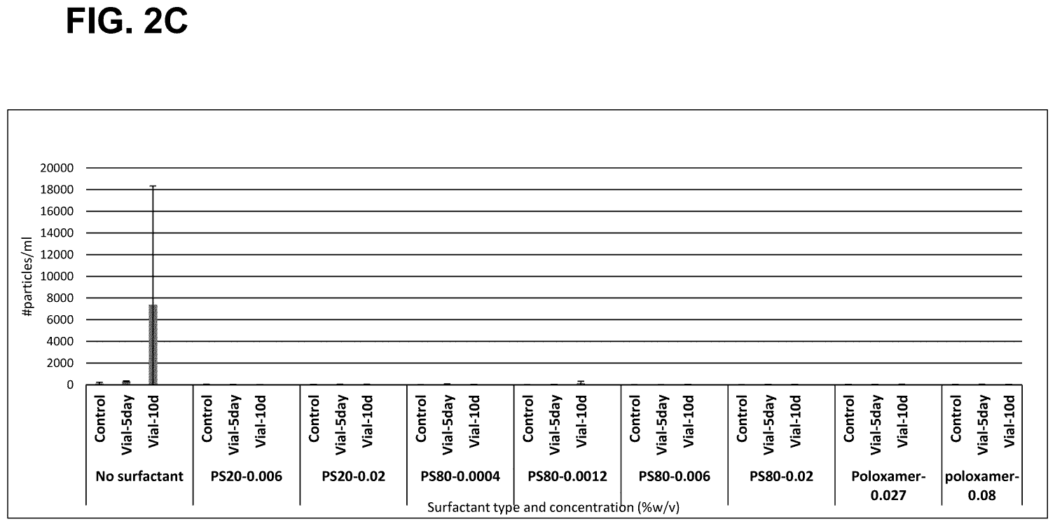

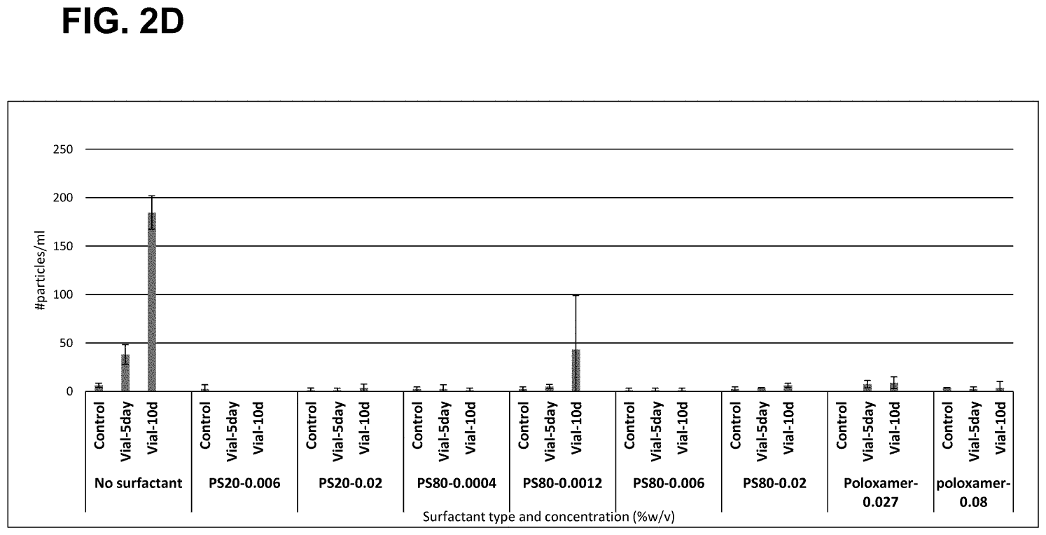

FIGS. 2A-2D show that formulations in glass vials containing no surfactant had significant presence of subvisible particles (≥2 μm, ≥5 μm, ≥10 μm, and ≥25 μm, respectively) after 5 days and 10 days of agitation. As expected, formulations in glass vials containing an amount of surfactant above CMC (0.02% PS20, 0.006% PS80, and 0.02% PS80) did not have a significant presence of subvisible particles after 5 days and 10 days of agitation. However, formulations in glass vials containing an amount of surfactant below CMC (0.006% PS20, 0.0004% PS80, 0.0012% PS80, 0.027% poloxamer 188, and 0.08% poloxamer 188) also did not have a significant presence of subvisible particles after 5 days and 10 days of rotation.

The same formulations were also tested for the presence of subvisible particles following freeze-thaw cycles. Prefilled vials containing the formulations were frozen in a −40° C. chamber and thawed to 25° C. seven times. Each freeze-thaw cycle lasted 1.5 hour. Following the last freeze-thaw cycle, formulations were tested for the presence of subvisible particles (≥2 μm and ≥25 μm) using MFI, as explained above.

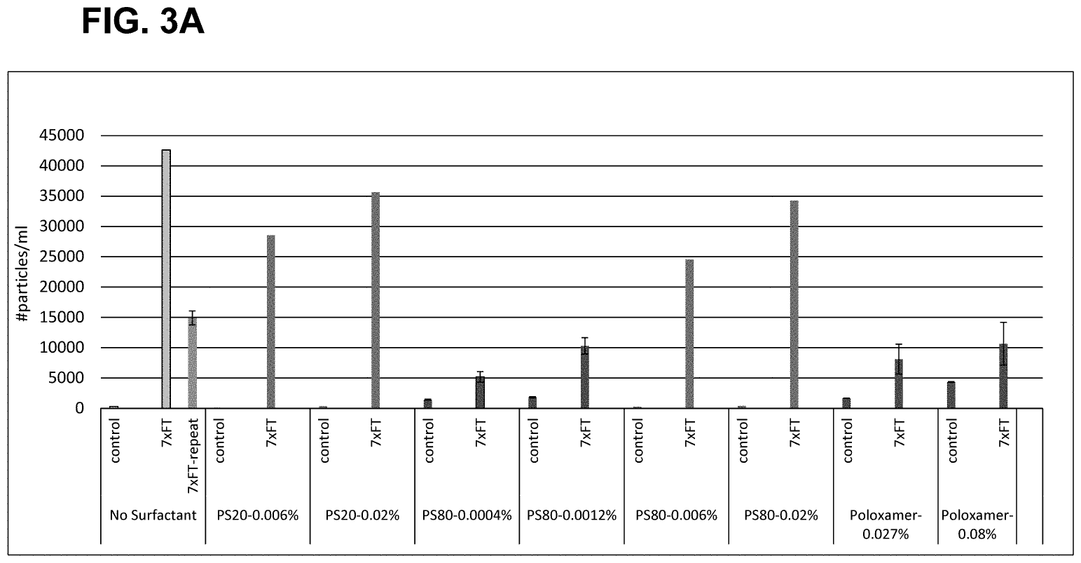

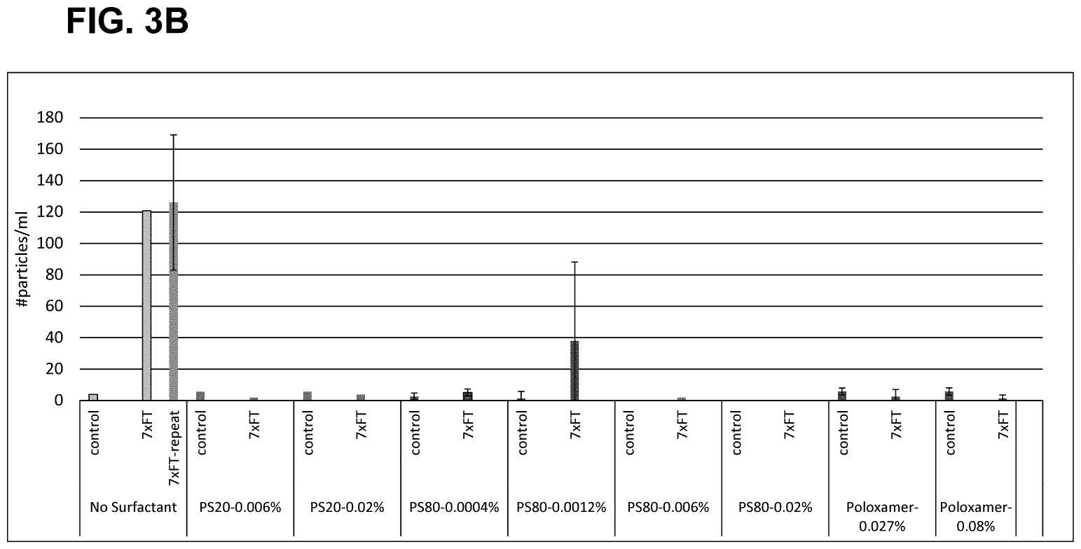

FIGS. 3A-3B show that formulations containing no surfactant had a significant presence of subvisible particles (≥2 μm and ≥25 μm, respectively) after seven freeze-thaw cycles (7×FT). As expected, formulations containing an amount of surfactant above CMC (0.02% PS20, 0.006% PS80, and 0.02% PS80) did not have a significant presence of subvisible particles after the freeze-thaw cycles. However, formulations containing an amount of surfactant below CMC (0.006% PS20, 0.0004% PS80, 0.0012% PS80, 0.027% poloxamer 188, and 0.08% poloxamer 188) also did not have a significant presence of subvisible particles after the freeze-thaw cycles.

### Example 2

Additional studies were performed to test the development of subvisible particles in anti-IL5R antibody formulations containing different surfactants, particularly, polysorbate 80 (PS80) or poloxamer 188 at an amount below the critical micelle concentration (CMC) of the surfactant.

Formulations were prepared containing 30 mg/mL anti-IL5R antibody, 20 mM histidine/histidine HCl, and 250 mM trehalose dihydrate, pH 6.0 with either no surfactant or 0.0004% polysorbate 80 (PS80), 0.0012% PS80, 0.02% poloxamer 188 (poloxamer), or 0.08% poloxamer 188. Anti-IL5R antibody was manufactured at DSC, MEDI-563 lot MS00684-42, PFB at 134 g/L. Using the source antibody, samples were reformulated by dilution and buffer exchange to achieve the desired concentrations. Surfactants were spiked from a liquid stock with a higher concentration. Final formulations were sterile filtered with 0.2 um filters.

For stability testing, 1 mL of the formulations was added to a syringe (Becton Dickinson Hypak 1 mL syringe, 29-gauge needle, with West 4023 plunger stopper) or glass vial (SCHOTT 2R type I glass vial, with West Daikyo 13 mm stopper) using manual filling and stoppering.

Aggressive agitation (end-to-end rotation) was then performed with a Scientific Industries Genie SI-1100 Roto-Shake Rotator/Rocker at the speed of 35 rpm for a total of 10 days. After agitation, syringes were emptied through the needle and formulation samples were tested with a MFI instrument, as explained in Example 1.

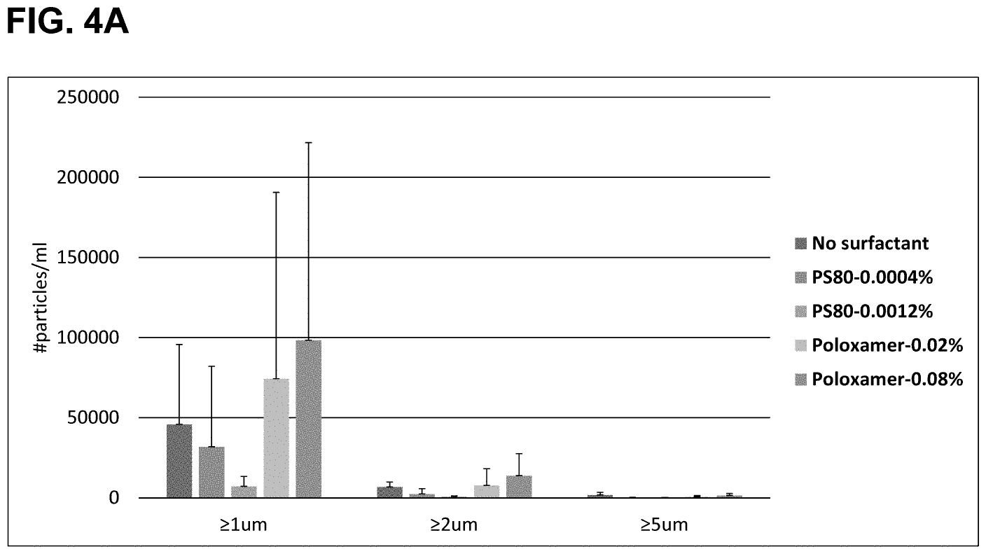

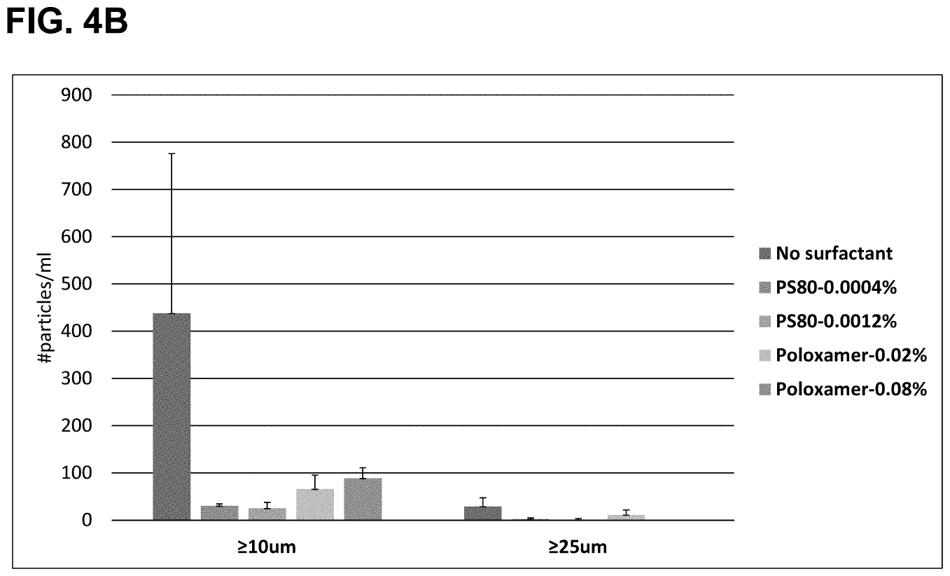

FIGS. 4A-4B show that formulations containing no surfactant had a significant presence of subvisible particles. Formulations containing an amount of surfactant below CMC (0.0004% PS80, 0.0012% PS80, 0.02% poloxamer 188 (poloxamer), and 0.08% poloxamer 188) did not have a significant presence of subvisible particles, particularly with regard to particles ≥5 μm, ≥10 μm, and ≥25 μm.

### Example 3

Additional studies were performed to test the development of subvisible particles in anti-IL5R antibody formulations containing different surfactants, particularly, polysorbate 80 (PS80) at amounts above and below the critical micelle concentration (CMC) of the surfactant.

Formulations were prepared containing 30 mg/mL anti-IL5R antibody, 20 mM histidine/histidine HCl, and 250 mM trehalose dihydrate, pH 6.0 with either no surfactant or 0.0004% polysorbate 80 (PS80), 0.0012% PS80, 0.006% PS80, or 002% PS80. Anti-IL5R antibody was manufactured at DSC, PFB at 134 g/L. Using the source antibody, samples were reformulated by dilution and buffer exchange to achieve the desired concentrations. Surfactants were spiked from a liquid stock with a higher concentration. Final formulations were sterile filtered with 0.2 um filters.

For stability testing, 1 mL of the formulations was added to a syringe (Becton Dickinson Hypak 1 mL syringe, 29-gauge needle, with West 4023 plunger stopper) or glass vial (SCHOTT 2R type I glass vial, with West Daikyo 13 mm stopper) using manual filling and stoppering.

Aggressive agitation (end-to-end rotation) was then performed with a Scientific Industries Genie SI-1100 Roto-Shake Rotator/Rocker at the speed of 35 rpm for a total of 10 days. After agitation, syringes were emptied through the needle and formulation samples were tested with a MFI instrument, as explained in Example 1.

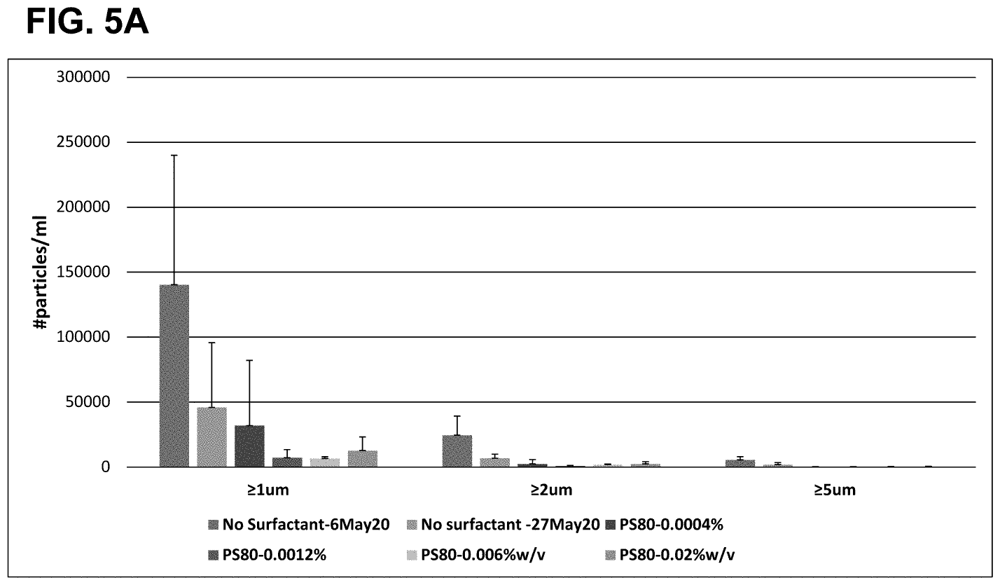

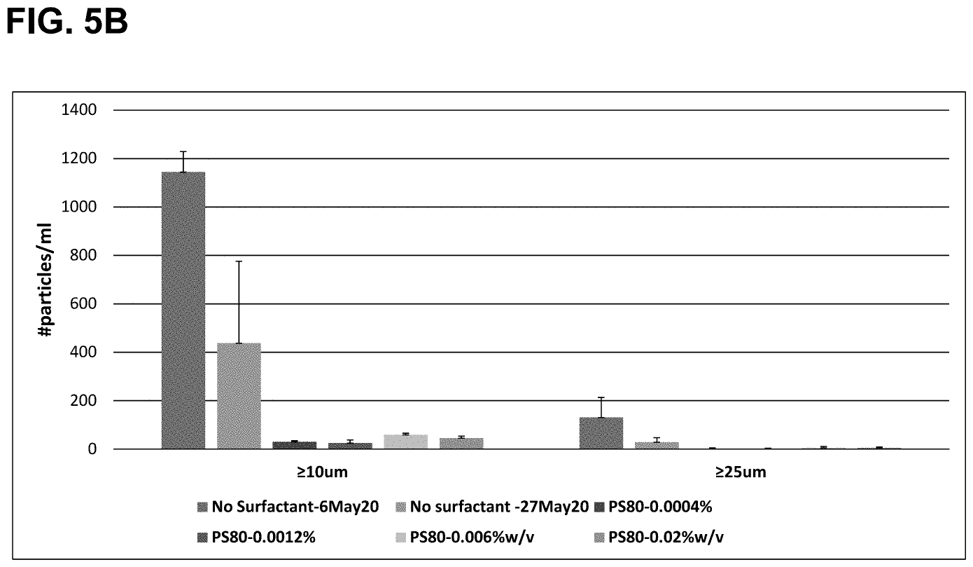

FIGS. 5A-5B show that formulations containing no surfactant had a significant presence of subvisible particles. As expected, formulations containing an amount of surfactant above CMC (0.006% PS80 and 0.02% PS80) did not have a significant presence of subvisible particles. Surprisingly, however, formulations containing an amount of surfactant below CMC (0.0004% PS80 and 0.0012% PS80) did not have a significant presence of subvisible particles, particularly with regard to particles having a diameter of 22 μm, ≥5 μm, ≥10 μm, and ≥25 μm.

### Example 4

Additional studies were performed to test the development of subvisible particles in anti-IL5R antibody formulations containing different surfactants, particularly, polysorbate 80 (PS80) and poloxamer 188 (poloxamer) at amounts above and below the critical micelle concentration (CMC) of the surfactant.

Formulations were prepared containing 10 mg/mL anti-IL5R antibody, 20 mM histidine/histidine HCl, and 250 mM trehalose dihydrate, pH 6.0 with either no surfactant, surfactant, or cyclodextrin, as explained in Table 1.

TABLE 1

| | CMC | MW | | below CMC | | above CMC | |
|---|---|---|---|---|---|---|---|
| Surfactant | (% w/v) | (g/mol) | Type | (% w/v) | | (% w/v) | |
| PS20 | 0.0074 | 1228 | non- | 0.002 | 0.006 | 0.020 | 0.050 |
| | | | ionic | | | | |
| PS80 | 0.0015 | 1310 | non- | 0.0004 | 0.0012 | 0.0041 | 0.0101 |
| | | | ionic | | | | |
| Poloxamer 188 | 0.10 | 8350 | non- | 0.0270 | 0.0811 | 0.2703 | 0.6757 |
| | | | ionic | | | | |
| Vitamin E TPGS 1000 | 0.02 | 1513 | non- | 0.0054 | 0.0162 | 0.0541 | 0.1351 |
| | | | ionic | | | | |
| Brij-35 | 0.011 | 1225 | non- | 0.0030 | 0.0089 | 0.0297 | 0.0743 |
| | | | ionic | | | | |
| | | | | lower | | higher | |
| | mid conc | MW | | concentration | | concentration | |
| Excipient | (% w/v) | (gr/mol) | Type | (% w/v) | | (% w/v) | |
| Hydroxypropyl-β- | 0.28 | 1135 | non- | 0.0767 | 0.2301 | 0.7669 | 1.9172 |
| cyclodextrin | | | ionic | | | | |

Anti-IL5R antibody was manufactured at DSC, PFB at 134 g/L. Using the source antibody, samples were reformulated by dilution and buffer exchange to achieve the desired concentrations. Surfactants were spiked from a liquid stock with a higher concentration. Final formulations were sterile filtered with 0.2 um filters.

For stability testing, 1 mL of the formulations were added to a glass vial (SCHOTT 2R type I glass vial, with West Daikyo 13 mm stopper) using manual filling and stoppering. Vials filled with formulations were stressed using a Lansmont Model 1000 transportation simulator to mimic agitation that can occur during truck and/or air transportation prior to being placed on stability at the ambient temperature. The agitation program used by the simulator followed the American Society of Testing and Materials D4169 (Standard Practice for Performance Testing of Shipping Containers and Systems) guideline. The simulated shipment was composed of two Level II truck-air-truck cycles over 12 hours. After shipment, vials were placed on stability upright at the intended storage condition of 2-8° C. and under accelerated 25° C. conditions, and sampled at 0, 0.25, 0.5, 1, 2, 3 and 6 months. MFI was used to measure the subvisible particle count, as explained in Example 1.

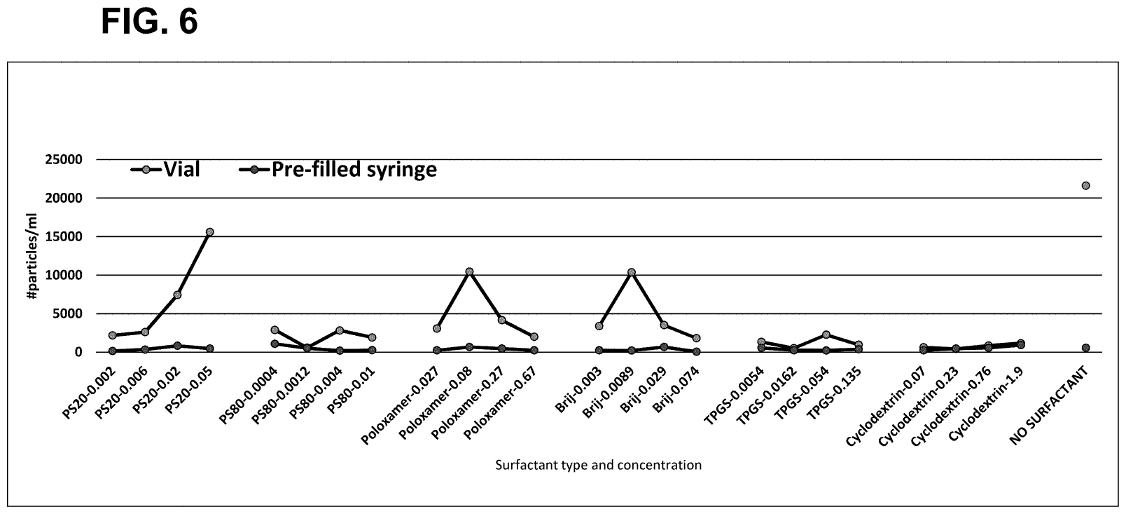

FIG. 6 shows that vial formulations containing no surfactant had a significant presence of subvisible particles (≥22 μm). Vial and prefilled syringe formulations containing an amount of surfactant above CMC (e.g., 0.004% PS80 and 0.01% PS80) did not have a significant presence of subvisible particles. However, surprisingly, vial and prefilled syringe formulations containing an amount of surfactant below CMC (e.g., 0.0004% PS80, 0.0012% PS80, 0.027% poloxamer, and 0.08% poloxamer) also did not have a significant presence of subvisible particles.

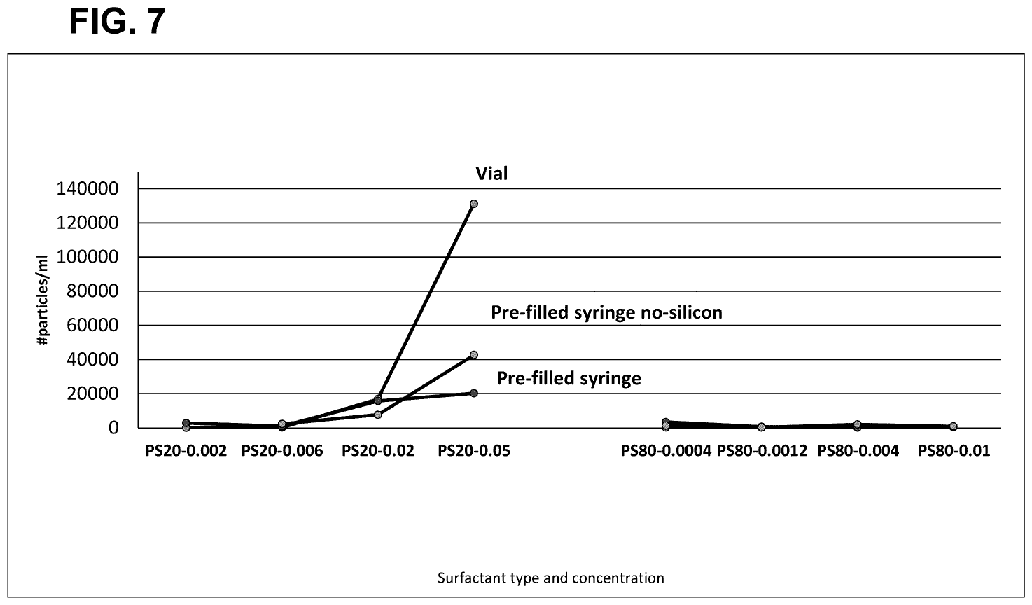

FIG. 7 further shows that formulations containing PS80 at concentrations above and below CMC in vials, prefilled syringes containing silicone, and prefilled syringe formulations not containing silicone did not have a significant presence of subvisible particles (≥22 μm).

### Example 5

Additional studies were performed to test the development of subvisible particles in anti-IL5R antibody formulations containing different combinations of buffer (acetate (pH 5.5) or succinate (pH 5.6)), 250 mM sugar (sucrose or mannitol), and surfactant (0.006% PS-20, 0.0004% PS-80, 0.0012% PS-80, 0.006% PS-80, 0.27% Poloxamer, 0.08% Poloxamer). The formulations (1 mL) were then subject to different stressors as described above (3 freeze-thaw cycles, 5 days of agitation, 10 days of agitation, or 7 freeze thaw cycles) in either a 2R glass vial or a 1 mL prefilled syringe. The following controls were used (i) Histidine-Trehalose—0.006% PS-20, (ii) Histidine-Trehalose—no surfactant, (iii) acetate-sucrose—no surfactant, (iv) acetate-mannitol—no surfactant, (v) succinate-sucrose—no surfactant, and (vi) succinate-mannitol—no surfactant.

Surfactant concentrations above and below CMC decrease subvisible particle formation after freeze-thaw cycles in glass vials containing formulations comprising acetate or succinate and sucrose or mannitol.

Surfactant concentrations above and below CMC decrease subvisible particle formation after agitation for 10 days (end-to-end rotation) in glass vials containing formulations comprising acetate or succinate and sucrose or mannitol.

Surfactant concentrations above and below CMC decrease subvisible particle formation after agitation for 10 days (end-to-end rotation) in prefilled syringes containing formulations comprising acetate or succinate and sucrose or mannitol.

Surfactant concentrations above and below CMC decrease subvisible particle formation after 7 freeze-thaw cycles in prefilled syringes containing formulations comprising acetate or succinate and sucrose or mannitol.

Formulation purity was also assessed using dynamic light scattering (DLS) (diffusion interaction parameters and diffusion coefficients).

### Example 6

In order to confirm that the raw excipients (buffers and sugars) used in the examples above did not have surface active impurities that could impact subvisible particle formation, surface tension measurements of the excipients used were measured. The results are shown in Table 2.

TABLE 2

| | Surface Tension | StDEV |
|---|---|---|
| Formulation | (mN/m) | (mN/m) |
| Water | 70.4 | 0.1 |
| Water-Trehalose-250 mM | 68.8 | 0.2 |
| Water-Mannitol-250 mM | 70.5 | 0.1 |
| Water-Sucrose-250 mM | 70.2 | 0.2 |
| Buffer-Histidine-Trehalose | 70.6 | 0.2 |
| Buffer-Succinate-Mannitol-pH 5.6 | 70.7 | 0.1 |
| Buffer-Succinate-Sucrose-pH 5.6 | 71.4 | 0.1 |
| Buffer-Acetate-Mannitol-pH 5.5 | 71.9 | 0.1 |
| Buffer-Acetate-Sucrose-pH 5.5 | 70.3 | 0.1 |
| Buffer-Phosphate-Mannitol-pH 6.5 | 71.5 | 0.1 |
| Buffer-Phosphate-Sucrose-pH 6.5 | 72.0 | 0.1 |

Overall, there was 1-2 mN/m assay variability, and the surface tension of water is reported as 72 mN/m in literature. However, within the assay, variability of all excipients and combinations had similar surface tension as water, and no significant drop (which would happen in presence of surfactants) was observed.

### Example 7

Subvisible particle formation was also assessed in formulations comprising 150 mg/ml anti-IL5R antibody. In these assays, the formulations were tested after 1 μm storage at 40° C. in 2R glass vials with 1 mL fill volume or after 1 μm storage at 5 C in 2R glass vials and Prefilled syringe with 1 mL fill volume. The formulations were stressed using a Lansmont Model 1000 transportation simulator 12 hours prior to storage. Formulations in vials at 40° C. comprising PS20, Brij, or cyclodextrin had a greater number of particles (≥2 μm). In addition, formulations at 5° C. comprising PS20 had higher number of particles (≥22 μm) than other formulations.

Those skilled in the art will recognize, or be able to ascertain using no more than routine experimentation, many equivalents to the specific aspects of the disclosure described herein. Such equivalents are intended to be encompassed by the following claims.

Although the foregoing aspects have been described in some detail by way of illustration and example for purposes of clarity of understanding, it will be obvious that certain changes and modifications can be practiced within the scope of the appended claims.

1. A pharmaceutical formulation, comprising:
(i) an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; and a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO: 5; and a light chain variable region CDR3 comprising the sequence set forth in SEQ ID NO: 6; and
(ii) a surfactant at an amount below the critical micelle concentration (CMC) of said surfactant, wherein the surfactant is not polysorbate 20 (PS20).

2. The pharmaceutical formulation of claim 1, wherein the surfactant is polysorbate 80 (PS80), poloxamer, a Brij series surfactant, or tocopheryl polyethylene glycol succinate (TPGS).

3. The pharmaceutical formulation of claim 1, wherein the antibody or antigen binding fragment thereof comprises a heavy chain variable region comprising SEQ ID NO:7 and a light chain variable region comprising SEQ ID NO:8.

4. The pharmaceutical formulation of claim 1, wherein the antibody or antigen binding fragment thereof comprises a heavy chain comprising SEQ ID NO:9 and a light chain comprising SEQ ID NO:10.

5. The pharmaceutical formulation of claim 1, comprising from about 2 mg/mL to about 200 mg/mL of the antibody or antigen binding fragment thereof.

6. The pharmaceutical formulation of claim 1, comprising from about 2 mg/mL to about 150 mg/mL of the antibody or antigen binding fragment thereof, optionally comprising about 150 mg/mL of the antibody or antigen binding fragment thereof.

7. The pharmaceutical formulation of claim 1, comprising from about 2 mg/mL to about 100 mg/mL of the antibody or antigen binding fragment thereof.

8. The pharmaceutical formulation of claim 1, comprising about 30 mg/mL of the antibody or antigen binding fragment thereof.

9. The pharmaceutical formulation of claim 1, wherein the amount of surfactant is at least about 10% below, at least 20% below, at least 30% below, at least 40% below, at least 50% below, at least 60% below, at least 70% below, at least 80% below, at least 90% below, at least 95% below, at least 96% below, at least 97% below, at least 98% below, or at least 99% below the CMC of said surfactant.

10. The pharmaceutical formulation of claim 1, wherein the surfactant is PS80 at an amount of from about 0.0004% (w/v) to about 0.0012% (w/v), optionally at 0.0004% (w/v), 0.0012% (w/v), or 0.006% (w/v).

11. The pharmaceutical formulation of claim 1, wherein the surfactant is TPGS at an amount of from about 0.001% (w/v) to about 0.02% (w/v).

12. The pharmaceutical formulation of claim 1, wherein the surfactant is a Brij series surfactant at an amount of from about 0.001% (w/v) to about 0.01% (w/v).

13. The pharmaceutical formulation of claim 1, wherein the surfactant is PS20 at an amount of about 0.006% (w/v).

14. The pharmaceutical formulation of claim 1, wherein the surfactant is poloxamer at an amount of from about 0.027% (w/v) to about 0.08% (w/v).

15. The pharmaceutical formulation of claim 1, wherein the surfactant is poloxamer at an amount of from about 0.03% (w/v) to about 0.08% (w/v).

16. The pharmaceutical formulation of claim 14, wherein the poloxamer is poloxamer 188.

17. The pharmaceutical formulation of claim 16, further comprising (iii) an uncharged excipient.

18. The pharmaceutical formulation of claim 17, comprising from about 20 mM to about 80 mM of the uncharged excipient.

19. The pharmaceutical formulation of claim 17, comprising from about 200 mM to about 400 mM of the uncharged excipient.

20. The pharmaceutical formulation of claim 19, comprising from about 1.5% (w/v) to about 8.5% (w/v) of the uncharged excipient.

21. The pharmaceutical formulation of claim 20, wherein the uncharged excipient is fructose, glucose, mannose, sorbose, xylose, lactose, maltose, sucrose, dextran, pullulan, dextrin, cyclodextrin, soluble starch, trehalose, sorbitol, erythritol, isomalt, lactitol, maltitol, xylitol, glycerol, lactitol, hydroxyethyl starch, water-soluble glucan, or a combination thereof.

22. The pharmaceutical formulation of claim 21, wherein the uncharged excipient is sucrose, optionally about 250 mM sucrose.

23. The pharmaceutical formulation of claim 21, wherein the uncharged excipient is trehalose.

24. The pharmaceutical formulation of claim 20, wherein the uncharged excipient is mannitol, optionally about 250 mM mannitol.

25. The pharmaceutical formulation of claim 24, further comprising (iv) arginine.

26. The pharmaceutical formulation of claim 25, further comprising (v) histidine.

27. The pharmaceutical formulation of claim 26, comprising from about 15 mM to about 30 mM histidine.

28. The pharmaceutical formulation of claim 27, further comprising a buffer.

29. The pharmaceutical formulation of claim 28, wherein the buffer is acetate.

30. The pharmaceutical formulation of claim 28, wherein the buffer is succinate.

31. The pharmaceutical formulation of claim 30, wherein the pH of the formulation is about 5.5 to about 6.0.

32. The pharmaceutical formulation of claim 31, wherein the pH of the formulation is about 5.5.

33. The pharmaceutical formulation of claim 31, wherein the pH of the formulation is about 5.6.

34. A pharmaceutical formulation, comprising:
(i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6; and
(ii) about 0.0004% (w/v) to about 0.0012% (w/v) PS80, or about 0.03% (w/v) to about 0.08% (w/v) poloxamer.

35. The pharmaceutical formulation of claim 34, further comprising:
(iii) about 1.5% (w/v) to about 8.5% (w/v) trehalose;
(iv) about 100 mM to about 200 mM L-arginine; and
(v) about 15 mM to about 30 mM histidine.

36. A pharmaceutical formulation, comprising:
(i) about 2 mg/mL to about 100 mg/mL of an antibody or antigen binding fragment thereof comprising a heavy chain variable region CDR1 comprising the sequence set forth in SEQ ID NO:1; a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6;
(ii) about 0.0004% (w/v) PS80, about 0.0012% (w/v) PS80, about 0.03% (w/v) poloxamer or about 0.08% (w/v) poloxamer;
(iii) about 250 mM trehalose;
(iv) about 130 mM L-arginine; and
(v) about 20 mM histidine.

37. A pharmaceutical formulation, comprising:
(i) about 30 mg/mL of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6;
(ii) about 0.0004% (w/v) PS80, about 0.0012% (w/v) PS80, about 0.03% (w/v) poloxamer 188, or about 0.08% (w/v) poloxamer 188;
(iii) about 250 mM trehalose;
(iv) about 130 mM L-arginine; and
(v) about 20 mM histidine.

38. A pharmaceutical formulation, comprising:
(i) about 2 mg/mL to about 200 mg/mL of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6;
(ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer;
(iii) about 250 mM sucrose; and
(iv) acetate.

39. A pharmaceutical formulation, comprising:
(i) about 2 mg/mL to about 200 mg/mL of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6;
(ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer;
(iii) about 250 mM sucrose; and
(iv) succinate.

40. A pharmaceutical formulation, comprising:
(i) about 2 mg/mL to about 200 mg/mL of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6;
(ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer;
(iii) about 250 mM mannitol; and
(iv) acetate.

41. A pharmaceutical formulation, comprising:
(i) about 2 mg/mL to about 200 mg/mL of an antibody or antigen binding fragment thereof comprising a heavy chain variable region CDR1 comprising the sequence set forth in SEQ ID NO:1 a heavy chain variable region CDR2 comprising the sequence set forth in SEQ ID NO:2; a heavy chain variable region CDR3 comprising the sequence set forth in SEQ ID NO:3; a light chain variable region CDR1 comprising the sequence set forth in SEQ ID NO:4; a light chain variable region CDR2 comprising the sequence set forth in SEQ ID NO:5; and a light chain variable region CDR3 comprising the sequence set forth in SEQ ID NO:6;
(ii) about 0.006% (w/) PS20, about 0.0004% (w/v) PS80, about 0.0012% (w/v) PS80, about 0.006% (w/v) PS80, about 0.08% (w/v) poloxamer, or about 0.27% (w/v) poloxamer;
(iii) about 250 mM mannitol; and
(iv) succinate.

42. The pharmaceutical formulation of claim 38 comprising about 150 mg/mL of the antibody or antigen binding fragment thereof.

43. The pharmaceutical formulation of claim 38 comprising about 2 mg/mL to about 100 mg/mL of the antibody or antigen binding fragment thereof

44. The pharmaceutical formulation of claim 38 comprising about 30 mg/mL of the antibody or antigen binding fragment thereof.

45. The pharmaceutical formulation of claim 44, wherein the pharmaceutical formulation is stable following aggressive agitation for about 10 days at room temperature.

46. The pharmaceutical formulation of claim 45, wherein the pharmaceutical formulation has less than 20 subvisible particles per mL.

47. The pharmaceutical formulation of claim 46, wherein the pharmaceutical formulation has about 20% less, about 30% less, about 40% less, about 50% less, about 60% less, about 70% less, about 80% less, about 90% less, about 95% less, or about 99% less subvisible particles following from about 1 to about 7 freeze-thaw cycles.

48. The pharmaceutical formulation of claim 47, wherein the pharmaceutical formulation has about 20% less, about 30% less, about 40% less, about 50% less, about 60% less, about 70% less, about 80% less, about 90% less, about 95% less, or about 99% less subvisible particles following storage for about 1 month at about 40° C.

49. The pharmaceutical formulation of claim 48, suitable for intravenous, subcutaneous, or intramuscular administration.

50. The pharmaceutical formulation of claim 49, wherein the pharmaceutical formulation is a liquid pharmaceutical formulation.

51. The pharmaceutical formulation of claim 49, wherein the pharmaceutical formulation is a lyophilized pharmaceutical formulation.

52. A dosage form comprising the pharmaceutical formulation of claim 1 in a container.

53. The dosage form of claim 52, wherein the container is a plastic vial or glass vial.

54. The dosage form of claim 52, wherein the container is a pre-filled syringe.

55. The dosage form of claim 54, wherein the pre-filled syringe comprises a needle.

56. The dosage form of claim 55, wherein the needle is a 29 G thin wall needle.

57. The dosage form of claim 56, wherein the pre-filled syringe is a plastic syringe or a glass syringe.

58. The dosage form of claim 57, wherein the pre-filled syringe is coated with silicone.

59. A kit, comprising the pharmaceutical formulation of claim 1 and instructions for use.

60. A method of treating a pulmonary disease or disorder in a subject, comprising administering to the subject a therapeutically effective amount of the pharmaceutical formulation of claim 1.

61. The method of claim 60, wherein the pulmonary disease or disorder is an eosinophilic disease or disorder.

62. The method of claim 60, wherein the pulmonary disease or disorder is asthma, chronic obstructive pulmonary disease (COPD), allergic bronchopulmonary aspergillosis, acute and chronic eosinophilic bronchitis, acute and chronic eosinophilic pneumonia, Churg-Strauss syndrome, hypereosinophilic syndrome, drug, irritant and radiation-induced pulmonary eosinophilia, infection-induced pulmonary eosinophilia, autoimmune-related pulmonary eosinophilia, eosinophilic esophagitis, Crohn's disease, or a combination thereof.

63. The method of claim 60, wherein the pulmonary disease or disorder is asthma.

64. The method of claim 63, wherein the asthma is eosinophilic asthma, neutrophilic asthma, combined eosinophilic and neutrophilic asthma, or aspirin sensitive asthma.


SEQUENCE LISTING (10 sequences)

SEQ ID NO: 1 - protein, 5 aa, Artificial Sequence
  Synthetic peptide
SYVIH

SEQ ID NO: 2 - protein, 17 aa, Artificial Sequence
  Synthetic peptide
YINPYNDGTKYNERFKG

SEQ ID NO: 3 - protein, 12 aa, Artificial Sequence
  Synthetic peptide
EGIRYYGLLGDY

SEQ ID NO: 4 - protein, 11 aa, Artificial Sequence
  Synthetic peptide
GTSEDIINYLN

SEQ ID NO: 5 - protein, 7 aa, Artificial Sequence
  Synthetic peptide
HTSRLQS

SEQ ID NO: 6 - protein, 9 aa, Artificial Sequence
  Synthetic peptide
QQGYTLPYT

SEQ ID NO: 7 - protein, 121 aa, Artificial Sequence [sequence omitted]

SEQ ID NO: 8 - protein, 107 aa, Artificial Sequence [sequence omitted]

SEQ ID NO: 9 - protein, 451 aa, Artificial Sequence
  Synthetic peptide
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Synthetic peptide
DIQMTQSPSSLSASVGDRVTITCGTSEDIINYLNWYQQKPGKAPKLLIYHTSRLQSGVPSRFSGSGSGTDFTLTISSLQPEDFATYYCQQGYTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC